(19) United States
(12) Patent Application Publication
(10) Pub. No.: US 2007/0098715 A1
(43) Pub. Date: May 3, 2007

(54) Antibodies against the tenascin major antigens

(76) Inventors: Seth Ettenberg, Cambridge, MA (US); Stephanie Masterman, Vancouver (CA); William LaRochelle, Madison, CT (US); Haihong Zhong, Guilford, CT (US)

Correspondence Address: MINTZ, LEVIN, COHN, FERRIS, GLOVSKY & POPEO, P.C., ONE FINANCIAL CENTER, BOSTON, MA, 02111, US

(21) Appl. No.: 11/391,630

(22) Filed: Mar. 27, 2006

Related U.S. Application Data
(60) Provisional application No. 60/665,592, filed on Mar. 25, 2005.

(51) Int. Cl. A61K 39/395; G01N 33/567; C07K 16/28

(52) U.S. Cl. 424/143.1; 435/7.2; 530/388.22

(57) ABSTRACT

The invention described herein relates to antibodies directed to the antigen Ten-M2 and uses of such antibodies. In particular, there are provided fully human monoclonal antibodies directed to the antigen Ten-M2. Isolated polynucleotide sequences encoding, and amino acid sequences comprising, heavy and light chain immunoglobulin molecules, particularly sequences corresponding to contiguous heavy and light chain sequences spanning the framework regions (FR's) and/or complementarity determining regions (CDR's), specifically from FR1 through FR4 or CDR1 through CDR3, are provided. Hybridomas or other cell lines expressing such immunoglobulin molecules and monoclonal antibodies are also provided.

### RELATED APPLICATION

This Application claims the benefit of priority from U.S. Provisional Application, Ser. No. 60/665,592 filed Mar. 25, 2005 the content of which is incorporated herein in its entirety.

### BACKGROUND OF THE INVENTION

1. Field of the Invention

The present invention relates generally to antibodies that bind to Ten-M proteins, and uses of such antibodies. In particular, there are provided fully human monoclonal antibodies directed to the antigen Ten-M2. Nucleotide sequences encoding, and amino acid sequences comprising, heavy and light chain immunoglobulin molecules, particularly sequences corresponding to contiguous heavy and light chain sequences spanning the framework regions and/or complementarity determining regions (CDR's), specifically from FR1 through FR4 or CDR1 through CDR3, are provided. Hybridomas or other cell lines expressing such immunoglobulin molecules and monoclonal antibodies are also provided.

2. Description of the Related Art

The human Ten-M family of genes, also known as teneurins or hOdz, are a class of type II transmembrane proteins containing a short intracellular N-terminus followed by a transmembrane region, which is followed by eight EGF-like repeats, which are followed by a large globular domain on the extracellular side. The EGF-like repeats of Ten-M proteins are thought to mediate dimerization. The expression patterns of mouse and chicken homologues of Ten-M proteins, as well as in vitro models of cell migration, such as neurite outgrowth, have suggested a role in neural development. This may also involve binding to extracellular matrix proteins such as heparin, indicating a role as a cell adhesion molecule.

The structure and function of the Ten-M protein has previously been examined (e.g., Oohashi et al., J. Cell Biol., 145:563-577 (1999)). The various forms of the protein (e.g., M1, M2, M3, and M4) are generally 2700 to 2800 amino acids in length.

Ten-M2 dimerizes via the EGF domain. The Ten-M family has been shown to associate with PS2 integrins, and the extracellular domain is known to interact with heparin.

### SUMMARY OF THE INVENTION

In one embodiment, a fully human antibody that selectively binds to a Ten-M2 protein on a cell and affect Ten-M2 function is provided. In some embodiments, the fully human antibody, when administered to a patient, reduces the metastasis of a cancer in a patient. In one aspect, a fully human antibody that selectively binds to a Ten-M2 protein that is not connected to a cell is provided. In one embodiment, the antibody does not bind to other Ten-M homologues, such as Ten-M3 or Ten-M4.

In one aspect, a conjugated fully human antibody that binds to a Ten-M2 protein is provided. Attached to the antibody is an agent, and the binding of the antibody to a cell results in the delivery of the agent to the cell. In one embodiment, the above conjugated fully human antibody binds to an extracellular section of the Ten-M2 protein. In another embodiment, the antibody binds to an EGF-like repeat of the Ten-M2 protein. In another embodiment, the antibody and conjugated toxin are internalized by a cell that expresses a Ten-M2 protein. In another embodiment, the agent is a cytotoxic agent. In another embodiment, the agent is saporin.

In a preferred embodiment, antibodies described herein bind to Ten-M2 with very high affinities (K_{d}). For example a human, rabbit, mouse, chimeric or humanized antibody that is capable of binding Ten-M2 with a K_{d} less than, but not limited to, 10⁻⁵, 10⁻⁶, 10⁻⁷, 10⁻⁸, 10⁻⁹, 10⁻¹⁰, 10⁻¹¹, 10⁻¹², 10⁻¹³ or 10⁻¹⁴, M, or any range or value therein. Affinity and/or avidity measurements can be measured by KinExA® and/or BIACORE®, as described herein.

Another embodiment includes antibodies that cross-compete for binding to Ten-M2 with the fully human antibodies disclosed herein. For example, antibodies derived from the same germline V_{H} and/or V_{L} genes that are capable of neutralizing Ten-M2 may bind to the same relevant epitope on the target and be capable of cross-competing with each other. Antibodies of the present invention may be tested in competitive ELISAs and/or competitive BIAcore studies to determine cross-reactivity.

In another aspect, a composition comprising a monoclonal antibody or antigen-binding portion described herein and a pharmaceutically acceptable carrier is provided.

In another aspect, a kit for treating Ten-M2 related disorders comprising a Ten-M2 antibody and instructions for administering the Ten-M2 antibody to a subject is provided.

In another aspect, a method of reducing the metastasis of a cancer in a patient is provided. The method comprises administering a fully human antibody to a patient. The antibody binds to a Ten-M2 protein so as to prevent the Ten-M2 protein from binding to and thereby forming a duplex with a second Ten-M2 protein. The antibody thereby reduces the metastasis of a cancer in a patient.

In another aspect, a method of reducing the risk of metastasis of a cancer in a patient is provided. The method comprises administering a fully human antibody to a patient. The antibody binds to a first Ten-M2 protein in a manner so as to prevent the Ten-M2 protein from forming an active Ten-M2/Ten-M2 duplex with a second Ten-M2 protein. The antibody binds so as to still allow the first Ten-M2 protein to bind to the second Ten-M2 protein. The antibody thereby reduces the risk of metastasis of a cancer in a patient.

In another aspect, a method of selectively killing a cancerous cell in a patient is provided. The method comprises administering a fully human antibody conjugate to a patient. The fully human antibody conjugate comprises an antibody that can bind to a Ten-M2 protein and an agent. The agent is either a toxin or another substance that will kill a cancer cell. The antibody conjugate thereby selectively kills the cancer cell. The agent can be saporin.

In another aspect, a method of diagnosing a risk of cancer metastasis in a patient is provided. The method comprises administering to a patient a fully human antibody conjugate that selectively binds to a Ten-M2 protein on a cell. The antibody conjugate comprises an antibody that selectively binds to Ten-M2 and a label. The method further comprises observing the presence of the label in the patient. A relatively high amount of the label will indicate a relatively high risk of cancer metastasis and a relatively low amount of the label will indicate a relatively low risk of cancer metastasis. In one embodiment, the label is a green fluorescent protein.

In a different aspect, the invention includes a method for diagnosing a condition associated with the expression of Ten-M2 in a cell, comprising contacting the cell with an anti-Ten-M2 antibody, and detecting the presence of Ten-M2. Preferred conditions include, without limitation, cancer of the lung, kidney, ovary, and brain.

Further embodiments include an isolated antibody, or fragment thereof, that comprises a heavy chain amino acid sequence. Other embodiments include an isolated antibody, or fragment thereof, that comprises a heavy chain nucleic acid sequence.

In one aspect, the invention provides an isolated antibody that binds to Ten-M2 and has a heavy chain amino acid sequence selected from the group consisting of SEQ ID NOS: 2, 6, 10, 14, 18, 22, 26, 30, 34, 38, 42, 46, and 50.

In another aspect, the invention provides an isolated antibody that binds to Ten-M2 and that comprises a light chain amino acid sequence selected from the group consisting of SEQ ID NOs: 4, 8, 12, 16, 20, 24, 28, 32, 36, 40, 44, 48, 52.

In yet another aspect, the invention provides an isolated antibody that binds to Ten-M2 and that comprises a heavy chain amino acid sequence selected from the group consisting of SEQ ID NOs: 2, 6, 10, 14, 18, 22, 26, 30, 34, 38, 42, 46, and 50 and that comprises a light chain amino acid sequence selected from the group consisting of SEQ ID NOs: 4, 8, 12, 16, 20, 24, 28, 32, 36, 40, 44, 48, 52.

In one embodiment, the isolated antibodies are monoclonal antibodies. In another embodiment, the isolated antibodies are chimeric antibodies. In yet another embodiment, the isolated antibodies are human antibodies.

In another aspect, the invention provides an isolated antibody that binds to Ten-M2 and that comprises a heavy chain amino acid sequence comprising the following CDRs (as defined by Kabat et al., Sequences of Proteins of Immunological Interest, Fifth Edition, NIH Publication 91-3242, Bethesda, M.d. [1991], vols. 1-3): (a) CDR1 consisting of the sequence of amino acids 26 to 35 of SEQ ID NOs: 2, 6, 10, 14, 18, 22, 26, 30, 34, 38, 42, 46, and 50 (b) CDR2 consisting of the sequence of amino acids 50 to 66 of SEQ ID NOs: 2, 6, 10, 14, 18, 22, 26, 30, 34, 38, 42, 46, and 50 and (c) CDR3 consisting of the sequence of amino acids 99 to 111 of SEQ ID NO: 10, 18 or 42, or amino acids 99 to 105 of SEQ ID NO: 2, or amino acids 99 to 114 of SEQ ID NOs: 14 or 22, or amino acids 99 to 117 of SEQ ID NO: 6, or amino acids 99 to 110 of SEQ ID NOs: 26 or 30, or amino acids 99 to 109 of SEQ ID NOs: 34 or 38, or amino acids 99 to 112 of SEQ ID NOs: 46 or 50.

In yet another aspect, the invention provides an isolated antibody that binds to Ten-M2 and that comprises a light chain amino acid sequence comprising the following CDRs (as defined by Kabat et al., Sequences of Proteins of Immunological Interest, Fifth Edition, NIH Publication 91-3242, Bethesda, M.d. [1991], vols. 1-3): (a) CDR1 consisting of the sequence of amino acids 24 to 39 of any of SEQ ID NOs: 4, 16, 36 or 40, or amino acids 24 to 35 of any of SEQ ID NOs: 8, 24, 28 or 32 or amino acids 24 to 34 of SEQ ID NO: 12, 44, 48 or 52, or amino acids 24 to 40 of SEQ ID NO.: 20 (b) CDR2 consisting of the sequence of amino acids 55 to 61 of any of SEQ ID NOs: 4, 16, 36 or 40, or amino acids 51 to 57 of any of SEQ ID NOs: 8, 24, 28 or 32, or amino acids 50 to 56 of SEQ ID NO: 12, 44, 48 or 52, or amino acids 56 to 62 of SEQ ID NO: 20; and (c) CDR3 consisting of the sequence of amino acids 94 to 101 of SEQ ID NO: 4, or amino acids 90 to 98 of any of SEQ ID NOs: 8, 24, 28 or 32, or amino acids 89 to 96 of SEQ ID NO: 12, or amino acids 94 to 102 of SEQ ID NO: 16, 36 or 40, or amino acids 95 to 103 of SEQ ID NO: 20, or amino acids 89 to 97 of SEQ ID NO: 44, 48 or 52.

It will be appreciated that in these embodiments, the isolated antibodies can be monoclonal antibodies, chimeric antibodies and/or human or humanized antibodies. Preferably, the antibodies are human antibodies.

It will also be appreciated that embodiments of the invention are not limited to any particular form of an antibody. For example, the antibodies provided may be a full length antibody (e.g. having an intact human Fc region) or an antibody fragment (e.g. a Fab, Fab′ or F(ab′)₂). In addition, the antibodies may be manufactured from a hybridoma that secretes the antibody, or from a recombinantly produced cell that has been transformed or transfected with a gene or genes encoding the antibody.

Other embodiments of the invention include isolated nucleic acid .molecules encoding any of the anti-Ten-M2 antibodies described herein, vectors having an isolated nucleic acid molecule encoding the anti-Ten-M2 antibody, and a host cell transformed with such a nucleic acid molecule. In addition, one embodiment of the invention is a method of producing an anti-Ten-M2 antibody by culturing host cells under conditions wherein a nucleic acid molecule is expressed to produce the antibody followed by recovering the antibody from the host cell.

In yet further embodiments, the invention provides an isolated polynucleotide molecule described herein.

Another embodiment of the invention is a fully human antibody that binds to other Ten-m family members including Ten-M3, Ten-M4.

In yet another aspect, the invention includes a method for inhibiting cell proliferation associated with the expression of Ten-M2, comprising treating cells expressing Ten-M2 with an effective amount of an anti-Ten-M2 antibody. In another aspect, the invention provides an article of manufacture comprising a container, comprising a composition containing an anti-Ten-M2 antibody, and a package insert or label indicating that the composition can be used to treat cancer characterized by the overexpression of Ten-M2. Preferably a mammal and, more preferably, a human, receives the anti-Ten-M2 antibody. In a preferred embodiment, tumors or cancers are treated, including, without limitation, cancers such as lung, colon, gastric, renal, prostate or ovarian carcinomas or NHL (Non-Hogkins Lymphoma).

In yet another aspect, the invention includes a method for treating diseases or conditions associated with the expression of Ten-M2 in a patient, comprising administering to the patient an effective amount of an anti-Ten-M2 antibody. The patient is a mammalian patient, preferably a human patient.

In a preferred embodiment, the method concerns the treatment of tumors including, without limitation, tumors of the lung, kidney, brain, and ovary, and certain types of gliomas and non-Hogkins lymphoma (NHL).

### BRIEF DESCRIPTION OF THE DRAWINGS

FIG. 1 is a depiction of the amino acid sequence of the Ten-M2 antigens used for immunization of XenoMouse®.

FIG. 2 is a binding FACS profile of Ten-M2 antibodies to SNB-19 cell line (positive for Ten-M2 expression) and IGROV-1 (negative for Ten-M2 expression), indicating Ten-M2 specificity of antibodies.

FIG. 3 is a bar graph showing inhibition of proliferation by Ten-M2 antibody drug conjugates on SNB- 19 cells.

FIG. 4 is a bar graph showing inhibition of proliferation by Ten-M2 antibody drug conjugates on IGROV- 1 cells.

FIG. 5 is a series of representations of the heavy chain and light chain variable region nucleotide and amino acid sequences of the human anti-Ten-M2 antibody designated Ten-M2-7.1.1 of the invention, with FIG. 5A representing the nucleotide sequence encoding the variable region of the heavy chain (SEQ ID NO:1), FIG. 5B representing the amino acid sequence (SEQ ID NO:2) encoded by the nucleotide sequence shown in FIG. 5A, FIG. 5C representing the nucleotide sequence encoding the variable region of the light chain (SEQ ID NO:3), and FIG. 5D representing the amino acid sequence (SEQ ID NO:4) encoded by the nucleotide sequence shown in FIG. 5C.

FIG. 6 is a series of representations of the heavy chain and light chain variable region nucleotide and amino acid sequences of the human anti-Ten-M2 antibody designated Ten-M2-7.2.1 of the invention, with FIG. 6A representing the nucleotide sequence encoding the variable region of the heavy chain (SEQ ID NO:5), FIG. 6B representing the amino acid sequence (SEQ ID NO:6) encoded by the nucleotide sequence shown in FIG. 6A, FIG. 6C representing the nucleotide sequence encoding the variable region of the light chain (SEQ ID NO:7), and FIG. 6D representing the amino acid sequence (SEQ ID NO:8) encoded by the nucleotide sequence shown in FIG. 6C.

FIG. 7 is a series of representations of the heavy chain and light chain variable region nucleotide and amino acid sequences of the human anti-Ten-M2 antibody designated Ten-M2-7.3.1 of the invention, with FIG. 7A representing the nucleotide sequence encoding the variable region of the heavy chain (SEQ ID NO:9), FIG. 7B representing the amino acid sequence (SEQ ID NO:10) encoded by the nucleotide sequence shown in FIG. 7A, FIG. 7C representing the nucleotide sequence encoding the variable region of the light chain (SEQ ID NO: 11), and FIG. 7D representing the amino acid sequence (SEQ ID NO: 12) encoded by the nucleotide sequence shown in FIG. 7C.

FIG. 8 is a series of representations of the heavy chain and light chain variable region nucleotide and amino acid sequences of the human anti-Ten-M2 antibody designated Ten-M2-7.7.1 of the invention, with FIG. 8A representing the nucleotide sequence encoding the variable region of the heavy chain (SEQ ID NO:13), FIG. 8B representing the amino acid sequence (SEQ ID NO:14) encoded by the nucleotide sequence shown in FIG. 8A, FIG. 8C representing the nucleotide sequence encoding the variable region of the light chain (SEQ ID NO:15), and FIG. 8D representing the amino acid sequence (SEQ-ID NO:16) encoded by the nucleotide sequence shown in FIG. 8C.

FIG. 9 is a series of representations of the heavy chain and light chain variable region nucleotide and amino acid sequences of the human anti-Ten-M2 antibody designated Ten-M2-8.1 of the invention, with FIG. 9A representing the nucleotide sequence encoding the variable region of the heavy chain (SEQ ID NO: 17), FIG. 9B representing the amino acid sequence (SEQ ID NO: 18) encoded by the nucleotide sequence shown in FIG. 9A, FIG. 9C representing the nucleotide sequence encoding the variable region of the light chain (SEQ ID NO: 19), and FIG. 9D representing the amino acid sequence (SEQ ID NO:20) encoded by the nucleotide sequence shown in FIG. 9C.

FIG. 10 is a series of representations of the heavy chain and light chain variable region nucleotide and amino acid sequences of the human anti-Ten-M2 antibody designated Ten-M2-8.6 of the invention, with FIG. 10A representing the nucleotide sequence encoding the variable region of the heavy chain (SEQ ID NO:21), FIG. 10B representing the amino acid sequence (SEQ ID NO:22) encoded by the nucleotide sequence shown in FIG. 10A, FIG. 10C representing the nucleotide sequence encoding the variable region of the light chain (SEQ ID NO:23), and FIG. 10D representing the amino acid sequence (SEQ ID NO:24) encoded by the nucleotide sequence shown in FIG. 10C.

FIG. 11 is a series of representations of the heavy chain and light chain variable region nucleotide and amino acid sequences of the human anti-Ten-M2 antibody designated Ten-M2-120 of the invention, with FIG. 11A representing the nucleotide sequence encoding the variable region of the heavy chain (SEQ ID NO:25), FIG. 11B representing the amino acid sequence (SEQ ID NO:26) encoded by the nucleotide sequence shown in FIG. 11A, FIG. 11C representing the nucleotide sequence encoding the variable region of the light chain (SEQ ID NO:27), and FIG. 11D representing the amino acid sequence (SEQ ID NO:28) encoded by the nucleotide sequence shown in FIG. 11C.

FIG. 12 is a series of representations of the heavy chain and light chain variable region nucleotide and amino acid sequences of the human anti-Ten-M2 antibody designated Ten-M2-140 of the invention, with FIG. 12A representing the nucleotide sequence encoding the variable region of the heavy chain (SEQ ID NO:29), FIG. 12B representing the amino acid sequence (SEQ ID NO:30) encoded by the nucleotide sequence shown in FIG. 12A, FIG. 12C representing the nucleotide sequence encoding the variable region of the light chain (SEQ ID NO:31), and FIG. 12D representing the amino acid sequence (SEQ ID NO:32) encoded by the nucleotide sequence shown in FIG. 12C.

FIG. 13 is a series of representations of the heavy chain and light chain variable region nucleotide and amino acid sequences of the human anti-Ten-M2 antibody designated Ten-M2-171of the invention, with FIG. 13A representing the nucleotide sequence encoding the variable region of the heavy chain (SEQ ID NO:33), FIG. 13B representing the amino acid sequence (SEQ ID NO:34) encoded by the nucleotide sequence shown in FIG. 13A, FIG. 13C representing the nucleotide sequence encoding the variable region of the light chain (SEQ ID NO:35), and FIG. 13D representing the amino acid sequence (SEQ ID NO:36) encoded by the nucleotide sequence shown in FIG. 13C.

FIG. 14 is a series of representations of the heavy chain and light chain variable region nucleotide and amino acid sequences of the human anti-Ten-M2 antibody designated Ten-M2-179 of the invention, with FIG. 14A representing the nucleotide sequence encoding the variable region of the heavy chain (SEQ ID NO:37), FIG. 14B representing the amino acid sequence (SEQ ID NO:38) encoded by the nucleotide sequence shown in FIG. 14A, FIG. 14C representing the nucleotide sequence encoding the variable region of the light chain (SEQ ID NO:39), and FIG. 14D representing the amino acid sequence (SEQ ID NO:40) encoded by the nucleotide sequence shown in FIG. 14C.

FIG. 15 is a series of representations of the heavy chain and light chain variable region nucleotide and amino acid sequences of the human anti-Ten-M2 antibody designated Ten-M2-188 of the invention, with FIG. 15A representing the nucleotide sequence encoding the variable region of the heavy chain (SEQ ID NO:41), FIG. 15B representing the amino acid sequence (SEQ ID NO:42) encoded by the nucleotide sequence shown in FIG. 15A, FIG. 15C representing the nucleotide sequence encoding the variable region of the light chain (SEQ ID NO:43), and FIG. 15D representing the amino acid sequence (SEQ ID NO:44) encoded by the nucleotide sequence shown in FIG. 15C.

FIG. 16 is a series of representations of the heavy chain and light chain variable region nucleotide and amino acid sequences of the human anti-Ten-M2 antibody designated Ten-M2-199 of the invention, with FIG. 16A representing the nucleotide sequence encoding the variable region of the heavy chain (SEQ ID NO:45), FIG. 16B representing the amino acid sequence (SEQ ID NO:46) encoded by the nucleotide sequence shown in FIG. 16A, FIG. 16C representing the nucleotide sequence encoding the variable region of the light chain (SEQ ID NO:47), and FIG. 16D representing the amino acid sequence (SEQ ID NO:48) encoded by the nucleotide sequence shown in FIG. 16C.

FIG. 17 is a series of representations of the heavy chain and light chain variable region nucleotide and amino acid sequences of the human anti-Ten-M2 antibody designated Ten-M2-213 of the invention, with FIG. 17A representing the nucleotide sequence encoding the variable region of the heavy chain (SEQ ID NO:49), FIG. 17B representing the amino acid sequence (SEQ ID NO:50) encoded by the nucleotide sequence shown in FIG. 17A, FIG. 17C representing the nucleotide sequence encoding the variable region of the light chain (SEQ ID NO:51), and FIG. 17D representing the amino acid sequence (SEQ ID NO:52) encoded by the nucleotide sequence shown in FIG. 17C.

FIG. 18 is a table showing the alignment of the amino acid sequences of the heavy chain variable domain regions of twelve anti-Ten-M2 antibodies with their respective germline sequences. The “-” indicates identity with the germline sequence. Differences from germline due to somatic hypermutation are shown by the respective amino acid. The CDRs (CDR1, CDR2, CDR3) and FRs (FR1, FR2, and FR3) in the immunoglobulins are shown under the respective column headings.

FIG. 19 is a table showing the alignment of the amino acid sequences of the light chain variable domain regions of twelve anti-Ten-M2 antibodies with their respective germline sequences. The “-” indicates identity with the germline sequence. Differences from germline due to somatic hypermutation are shown by the respective amino acid. The CDRs (CDR1, CDR2, CDR3) and FRs (FR1, FR2, and FR3) in the immunoglobulins are shown under the respective column headings.

FIG. 20 is a Western Blot showing that anti-Ten-M2 antibodies specifically recognize the p125 Ten-M2 (lane M2) protein.

FIG. 21 is a Western Blot showing endogenous Ten-M2 protein in IGROV, SK-OV-3, SNB-19 and 786-0 cells using anti-Ten-M2 rabbit polyclonal antibody (upper panel) or anti-Ten-M2 monoclonal antibody clone 140 (lower panel).

FIG. 22 is immunohistochemical analysis of anti-Ten-M2 antibodies. FIG. 22A depicts Ten-M2 antibody staining on the membrane and cytoplasm of tumor cells in a breast cancer sample and in an isotype control. 10 of 12 samples stained positive. FIG. 22B depicts Ten-M2 antibody staining on the cytoplasm of tumor cells in an ovarian cancer sample and on an isotype control. 10 of 10 samples stained positive. FIG. 22C depicts Ten-M2 antibody staining on the membrane and cytoplasm of tumor cells in kidney carcinoma and on an isotype control. 9 of 9 samples stained positive. FIG. 22D depicts Ten-M2 antibody staining on the cytoplasm of tumor cells in a colon cancer sample and in an isotype control. 7 of 10 samples stained positive. FIG. 22E depicts Ten-M2 antibody staining on the cytoplasm of tumor cells and inflammation cells in a lung cancer sample and on an isotype control. 10 of 10 samples stained positive. FIG. 22F depicts Ten-M2 antibody staining on the cytoplasm of tumor cells and the endothelium in melanoma and on an isotype control. 10 of 10 samples stained positive. FIG. 22G depicts Ten-M2 antibody staining on the cytoplasm of tumor cells and the stroma in prostate cancer and on an isotype control. 10 of 10 samples stained positive. FIG. 22H depicts Ten-M2 antibody staining in the cytoplasm of normal tubular cells of the kidney and in an isotype control. FIG. 22I depicts Ten-M2 antibody staining on the membrane and cytoplasm of epithelium and stroma in a normal prostate sample and an isotype control.

FIG. 23 depicts bar graphs showing anti-Ten-M2-vcMMAE and anti-Ten-M2-MMAF in vitro growth inhibition. FIG. 23A is a bar graph showing anti-Ten-M2 mAb drug conjugates killing SNB-19 brain carcinoma cells. FIG. 23B is a bar graph showing anti-Ten-M2 mAB drug conjugates killing RXF-393 renal cell carcinoma cells. FIG. 23C is a bar graph showing anti-Ten-M2 mAB drug conjugates killing RXF-631 renal cell carcinoma cells. FIG. 23D is a bar graph showing anti-Ten-M2 mAB drug conjugates killing 786-0 renal cell carcinoma cells.

### DETAILED DESCRIPTION OF THE PREFERRED EMBODIMENT

Messenger RNA levels of human Ten-M proteins may be upregulated in certain cancers. Thus, Ten-M2 proteins may have a role in cell migration during cancer metastasis. Given the developmental studies suggesting a role in cell migration and the Ten-M proteins' expression in human cancer, it is possible that therapies designed towards Ten-M may inhibit metastasis of primary tumors. The administration of antibodies to the duplex forming regions of Ten-M2 proteins can thereby result in the inhibition of cancer metastasis.

Additionally, the Ten-M proteins appear to be involved in neuron growth and guidance. As such, the present compositions and methods can be applied to promote or inhibit neuron growth and development in situations where such needs arise. For example, the presently disclosed antibodies that promote neuron growth through the binding of the antibody to the Ten-M protein can be used in situations such as nerve regeneration in damaged tissues.

Some embodiments of the invention relate to the generation and identification of isolated, preferably fully human, monoclonal antibodies that bind to the Ten-M2 protein. In some embodiments, these antibodies can bind to Ten-M2 with high affinity, high potency, or both.

In some embodiments, these antibodies are associated with a toxin or similar compound and be used to associate the toxin to a particular location or cell type to promote the killing of the cell type or cells in a desired location. In some embodiments the antibodies are internalized with a high degree of efficiency.

In some embodiments, the antibody will prevent effective functioning (e.g., signaling) of the Ten-M2 protein. For example, the antibody may prevent dimerization of the Ten-M2 protein with another Ten-M2 protein by binding to a dimerization domain (e.g., EGF-like repeats) of the protein. Accordingly, some of the present embodiments provide isolated antibodies, or fragments of those antibodies, that bind to the EGF-like repeat domain of the Ten-M2 protein.

Embodiments of the invention also provide cells for producing these antibodies. In addition, embodiments provide for using these antibodies as a diagnostic or treatment for diseases related to the under or over expression of the Ten-M2 protein.

The nucleic acids described herein, and fragments and variants thereof, may be used, by way of nonlimiting example, (a) to direct the biosynthesis of the corresponding encoded proteins, polypeptides, fragments and variants as recombinant or heterologous gene products, (b) as probes for detection and quantification of the nucleic acids disclosed herein, (c) as sequence templates. Such uses are described more fully below.

Definitions:

Unless otherwise defined, scientific and technical terms used in connection with the present invention shall have the meanings that are commonly understood by those of ordinary skill in the art. Further, unless otherwise required by context, singular terms shall include pluralities and plural terms shall include the singular. The definitions provided herein control over definitions from external sources, including definitions provided in references which are incorporated by reference.

Generally, nomenclatures utilized in connection with, and techniques of, cell and tissue culture, molecular biology, and protein and oligo- or polynucleotide chemistry and hybridization described herein are those well known and commonly used in the art, as described in various general and more specific references such as those that are cited and discussed throughout the present specification. See e.g. Singleton et al., Dictionary of Microbiology and Molecular Biology 2^{nd} ed., J. Wiley & Sons (New York, N.Y. 1994); Sambrook et al. Molecular Cloning: A Laboratory Manual (2d ed., Cold Spring Harbor Laboratory Press, Cold Spring Harbor, N.Y. (1989)), which is incorporated herein by reference. Standard techniques are used for recombinant DNA, oligonucleotide synthesis, and tissue culture and transformation (e.g., electroporation, lipofection). Enzymatic reactions and purification techniques are performed according to manufacturer's specifications or as commonly accomplished in the art or as described herein. Standard techniques are also used for chemical syntheses, chemical analyses, pharmaceutical preparation, formulation, and delivery, and treatment of patients.

As utilized in accordance with the present disclosure, the following terms, unless otherwise indicated, shall be understood to have the following meanings:

“Polymerase chain reaction” or “PCR” refers to a procedure or technique in which minute amounts of a specific piece of nucleic acid, RNA and/or DNA, are amplified as described in U.S. Pat. No. 4,683,195 issued Jul. 28, 1987. Generally, sequence information from the ends of the region of interest or beyond needs to be available, such that oligonucleotide primers can be designed; these primers will be identical or similar in sequence to opposite strands of the template to be amplified. The 5′ terminal nucleotides of the two primers can coincide with the ends of the amplified material. PCR can be used to amplify specific RNA sequences, specific DNA sequences from total genomic DNA, and cDNA transcribed from total cellular RNA, bacteriophage or plasmid sequences, etc. See generally Mullis et al., Cold Spring Harbor Symp. Quant. Biol. 51:263 (1987); Erlich, ed., PCR Technology (Stockton Pres, N.Y., 1989). A used herein, PCR is considered to be one, but not the only, example of a nucleic acid polymerase reaction method for amplifying a nucleic acid test sample comprising the use of a known nucleic acid as a primer and a nucleic acid polymerase to amplify or generate a specific piece of nucleic acid.

“Antibodies” (Abs) and “immunoglobulins” (Igs) are glycoproteins having the same structural characteristics. While antibodies exhibit binding specificity to a specific antigen, immunoglobulins include both antibodies and other antibody-like molecules which lack antigen specificity. Polypeptides of the latter kind are, for example, produced at low levels by the lymph system and at increased levels by myelomas.

“Native antibodies and immunoglobulins” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light chain variable domain is aligned with the variable domain of the heavy chain. Particular amino acid residues are believed to form an interface between the light- and heavy-chain variable domains (Chothia et al. J. Mol. Biol. 186:651 (1985; Novotny and Haber, Proc. Natl. Acad. Sci. U.S.A. 82:4592 (1985); Chothia et al., Nature 342:877-883 (1989)).

The term “antibody” herein is used in the broadest sense and specifically covers intact monoclonal antibodies, polyclonal antibodies, multispecific antibodies (e.g. bispecific antibodies) formed from at least two intact antibodies, and antibody fragments including Fab and F(ab)′2, so long as they exhibit the desired biological activity. The “light chains” of antibodies (immunoglobulins) from any vertebrate species can be assigned to one of two clearly distinct types, called κ and λ, based on the amino acid sequences of their constant domains. Binding fragments are produced by recombinant DNA techniques, or by enzymatic or chemical cleavage of intact antibodies. Binding fragments include Fab, Fab′, F(ab′)₂, Fv, and single-chain antibodies, as described in more detail below. An antibody other than a “bispecific” or “bifunctional” antibody is understood to have each of its binding sites identical.

Depending on the amino acid sequence of the constant domain of their heavy chains, intact antibodies can be assigned to different “classes.” There are five major classes of intact antibodies: IgA, IgD, IgE, IgG, and IgM, and several of these may be further divided into “subclasses” (isotypes), e.g., IgG1, IgG2, IgG3, IgG4, IgA, and IgA2. The heavy-chain constant domains that correspond to the different classes of antibodies are called α, δ, ε, γ, and μ, respectively. The subunit structures and three-dimensional configurations of different classes of immunoglobulins are well known.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature, 256:495 (1975), or may be made by recombinant DNA methods (see, e.g., U.S. Pat. No. 4,816,567). The “monoclonal antibodies” may also be isolated from phage antibody libraries using the techniques described in Clackson et al, Nature, 352:624-628 (1991) and Marks et al., J. Mol. Biol., 222:581-597 (1991), for example.

An “isolated” antibody is one that has been identified and separated and/or recovered from a component of its natural environment. Contaminant components of its natural environment are materials that would interfere with diagnostic or therapeutic uses for the antibody, and may include enzymes, hormones, and other proteinaceous or nonproteinaceous solutes. In preferred embodiments, the antibody will be purified (1) to greater than 95% by weight of antibody as determined by the Lowry method, and terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

A “neutralizing antibody” is an antibody molecule that is able to eliminate or significantly reduce an effector function of a target antigen to which it binds. Accordingly, a “neutralizing” Ten-M2 antibody is capable of eliminating or significantly reducing an effector function, such as Ten-M2 activity. In one embodiment, a neutralizing antibody will reduce an effector function by 1-10, 10-20, 20-30, 30-50, 50-70, 70-80, 80-90, 90-95, 95-99, 99-100%. In one embodiment, the Ten-M2 antibody inhibits function by inhibiting, to some extent, the dimerization of two Ten-M2 proteins. In another embodiment, the Ten-M2 antibody inhibits function by inhibiting, to some extent, the association of the dimerized Ten-M2 protein duplex with another protein. In one embodiment, the neutralizing antibody inhibits dimmer formation by directly binding to the location on the Ten-M2 protein that binds to a second Ten-M2 protein. In another embodiment, the neutralizing antibody binds to one part of the Ten-M2 protein, while a part of the antibody, or something associated with the antibody, blocks the dimerization of the Ten-M2 proteins. In another embodiment, the antibody binds to the Ten-M2 protein and induces a conformational change in the protein which prevents dimerization from occurring.

In another embodiment, the Ten-M2 antibody actually increases the likelihood of dimerization occurring. In another embodiment, the antibodies are activating antibodies and binding of the antibody functions to effectively cause the Ten-M2 protein to act as if it had dimerized with another Ten-M2 protein.

“Antibody-dependent cell-mediated cytotoxicity” and “ADCC” refer to a cell-mediated reaction in which non-specific cytotoxic cells that express Ig Fc receptors (FcRs) (e.g. Natural Killer (NK) cells, neutrophils, and macrophages) recognize bound antibody on a target cell and subsequently cause lysis of the target cell. The primary cells for mediating ADCC, NK cells, express FcγRIII only, whereas monocytes express FcγRI, FcγRII and FcγRIII. FcRs expression on hematopoietic cells is summarized in Table 3 on page 464 of Ravetch and Kinet, Annu. Rev. Immunol 9:457-92 (1991). To assess ADCC activity of a molecule of interest, an in vitro.ADCC assay, such as that described in U.S. Pat. No. 5,500,362, or 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. PNAS (USA) 95:652-656 (1988).

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called complementarity-determining regions (CDRs) or hypervariable regions both in the Ig light-chain and heavy-chain variable domains. The more highly conserved portions of variable domains are called the framework (FR). The variable domains of native heavy and light chains each comprise four FR regions, largely adopting a β-sheet configuration, connected by three CDRs, which form loops connecting, and in some cases forming part of, the β-sheet structure. The CDRs in each chain are held together in close proximity by the FR regions and, with the CDRs from the other chain, contribute to the formation of the antigen-binding site of antibodies (see Kabat et al. (1991)). The constant domains are not involved directly in binding an antibody to an antigen, but exhibit various effector functions, such as participation of the antibody in antibody-dependent cellular toxicity.

Digestion of antibodies with the enzyme, papain, results in two identical antigen-binding fragments, known also as “Fab” fragments, and a “Fc” fragment, having no antigen-binding activity but having the ability to crystallize. Digestion of antibodies with the enzyme, pepsin, results in the a F(ab′)₂ fragment in which the two arms of the antibody molecule remain linked and comprise two-antigen binding sites. The F(ab′)₂ fragment has the ability to crosslink antigen.

“Fv” when used herein refers to the minimum fragment of an antibody that retains both antigen-recognition and antigen-binding sites.

“Fab” when used herein refers to a fragment of an antibody which comprises the constant domain of the light chain and the CHI domain of the heavy chain.

“Fv” is the minimum antibody fragment which contains a complete antigen-recognition and binding site. In a two-chain Fv species, this region consists of a dimer of one heavy- and one light-chain variable domain in tight, non-covalent association. In a single-chain Fv species, one heavy- and one light-chain variable domain can be covalently linked by a flexible peptide linker such that the light and heavy chains can associate in a “dimeric” structure analogous to that in a two-chain Fv species. It is in this configuration that the three CDRs of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The term “hypervariable region” when used herein refers to the amino acid residues of an antibody which are responsible for antigen-binding. The hypervariable region generally comprises amino acid residues from a “complementarity determining region” or “CDR” (e.g. residues 24-34 (L1), 50-62 (L2), and 89-97 (L3) in the light chain variable domain and 31-55 (Hi), 50-65 (H2) and 95-102 (H3) in the heavy chain variable domain; Kabat et al., Sequences of Proteins of Immunological Interest, 5^{th} Ed. Public Health Service, National Institutes of Health, Bethesda, Md. (1991)) and/or those residues from a “hypervariable loop” (e.g. residues 26-32 (L1), 50-52 (L2) and 91-96 (L3) in the light chain variable domain and 26-32 ((H1), 53-55 (H2) and 96-101 (H3) in the heavy chain variable domain; Chothia and Lesk J. Mol. Biol. 196:901-917 (1987)). “Framework Region” or “FR” residues are those variable domain residues other than the hypervariable region residues as herein defined.

The term “complementarity determining regions” or “CDRs” when used herein refers to parts of immunological receptors that make contact with a specific ligand and determine its specificity. The CDRs of immunological receptors are the most variable part of the receptor protein, giving receptors their diversity, and are carried on six loops at the distal end of the receptor's variable domains, three loops coming from each of the two variable domains of the receptor.

The term “epitope” is used to refer to binding sites for antibodies on protein antigens. Epitopic determinants usually consist of chemically active surface groupings of molecules such as amino acids or sugar side chains and usually have specific three dimensional structural characteristics, as well as specific charge characteristics. An antibody is said to bind an antigen when the dissociation constant is<1 μM, preferably≦100 nM and most preferably<10 nM. An increased or greater dissociation constant (“K_{D}”) means that there is less affinity between the epitope and the antibody. In other words, that the antibody and the epitope are less favorable to bind or stay bound together. A decrease of lower dissociation constant means that there is a higher affinity between the epitope and the antibody. In other words, it is more likely that the antibody and the epitope will bind or stay bound together. An antibody with a K_{D} of “no more than” a certain amount means that the antibody will bind to the epitope with the given affinity, or more strongly (or tightly).

While K_{D} describes the binding characteristics of an epitope and an antibody, “potency” describes the effectiveness of the antibody itself for a function of the antibody. A relatively low K_{D} does not automatically mean a high potency. Thus, antibodies can have a relatively low K_{D} and a high potency (e.g., they bind well and alter the function strongly), a relatively high K_{D} and a high potency (e.g., they don't bind well but have a strong impact on function), a relatively low K_{D} and a low potency (e.g., they bind well, but not in a manner effective to alter a particular function) or a relatively high K_{D} and a low potency (e.g., they simply do not bind to the target well). In one embodiment, high potency means that there is a high level of inhibition with a low concentration of antibody. In one embodiment, an antibody is potent or has a high potency when its IC₅₀ is a small value, for example, 1300-600, 600-200, 200-130, 130-120, 12-50, 50-10, 10-1 or less pM.

“Substantially,” unless otherwise specified in conjunction with another term, means that the value can vary within the any amount that is contributable to errors in measurement that may occur during the creation or practice of the embodiments. “Significant” means that the value can vary as long as it is sufficient to allow the claimed invention to function for its intended use.

The term “selectively bind” in reference to an antibody does not mean that the antibody only binds to a single substance. Rather, it denotes that the K_{D} of the antibody to a first substance is less than the K_{D} of the antibody to a second substance. Antibodies that exclusively bind to an epitope only bind to that single epitope.

The term “amino acid” or “amino acid residue,” as used herein, refers to naturally occurring L amino acids or to D amino acids as described further below with respect to variants. The commonly used one and three-letter abbreviations for amino acids are used herein (Bruce Alberts et al., Molecular Biology of the Cell, Garland Publishing, Inc., New York (3d ed. 1994)).

The term “mAb” refers to monoclonal antibody.

The term “XENOMOUSE® refers to strains of mice which have been engineered to contain 245 kb and 190 kb-sized germline configuration fragments of the human heavy chain locus and kappa light chain locus, as described in Green et al. Nature Genetics 7:13-21 (1994), incorporated herein by reference. The XENOMOUSE® strains are available from Abgenix, Inc. (Fremont, Calif.).

The term “XENOMAX®” refers use of to the use of the “Selected Lymphocyte Antibody Method” (Babcook et al., Proc. Natl. Acad. Sci. USA, i93:7843-7848 (1996)), when used with XENOMOUSE® animals.

The term “SLAM®@” refers to the “Selected Lymphocyte Antibody Method” (Babcook et al., Proc. Natl. Acad. Sci. USA, i93:7843-7848 (1996), and Schrader, U.S. Pat. No. 5,627,052), both of which are incorporated by reference in their entireties.

The terms “disease,” “disease state” and “disorder” refer to a physiological state of a cell or of a whole mammal in which an interruption, cessation, or disorder of cellular or body functions, systems, or organs has occurred.

The term “symptom” means any physical or observable manifestation of a disorder, whether it is generally characteristic of that disorder or not. The term “symptoms” can mean all such manifestations or any subset thereof.

The term “treat” or “treatment” refer to both therapeutic treatment and prophylactic or preventative measures, wherein the object is to prevent or slow down (lessen) an undesired physiological change or disorder, such as the development or spread of cancer. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented. The term “inhibit,” when used in conjunction with a disease or symptom can mean that the antibody can reduce or eliminate the disease or symptom.

The term “patient” includes human and veterinary subjects.

“Administer,” for purposes of treatment, means to deliver to a patient. For example and without limitation, such delivery can be intravenous, intraperitoneal, by inhalation, intramuscular, subcutaneous, oral, topical, transdermal, or surgical.

“Therapeutically effective amount,” for purposes of treatment, means an amount such that an observable change in the patient's condition and/or symptoms could result from its administration, either alone or in combination with other treatment.

A “pharmaceutically acceptable vehicle,” for the purposes of treatment, is a physical embodiment that can be administered to a patient. Pharmaceutically acceptable vehicles can be, but are not limited to, pills, capsules, caplets, tablets, orally administered fluids, injectable fluids, sprays, aerosols, lozenges, neutraceuticals, creams, lotions, oils, solutions, pastes, powders, vapors, or liquids. One example of a pharmaceutically acceptable vehicle is a buffered isotonic solution, such as phosphate buffered saline (PBS).

“Neutralize,” for purposes of treatment, means to partially or completely suppress chemical and/or biological activity.

“Down-regulate,” for purposes of treatment, means to lower the level of a particular target composition.

“Mammal” for purposes of treatment refers to any animal classified as a mammal, including humans, domestic and farm animals, and zoo, sports, or pet animals, such as monkeys, dogs, horses, cats, cows, etc.

The term “polynucleotide” as referred to herein means a polymeric form of nucleotides of at least 10 bases in length, either ribonucleotides or deoxynucleotides or a modified form of either type of nucleotide. The term includes single and double stranded forms of DNA.

The term “isolated polynucleotide” as used herein shall mean a polynucleotide of genomic, cDNA, or synthetic origin or some combination thereof, which by virtue of its origin the “isolated polynucleotide” (1) is not associated with all or a portion of a polynucleotide in which the “isolated polynucleotide” is found in nature, (2) is operably linked to a polynucleotide which it is not linked to in nature, or (3) does not occur in nature as part of a larger sequence.

The term “oligonucleotide” referred to herein includes naturally occurring, and modified nucleotides linked together by naturally occurring, and non-naturally occurring oligonucleotide linkages. Oligonucleotides are a polynucleotide subset generally comprising a length of 200 bases or fewer. Preferably, oligonucleotides are 10 to 60 bases in length and most preferably 12, 13, 14, 15, 16, 17, 18, 19, or 20 to 40 bases in length. Oligonucleotides are usually single stranded, e.g., for probes; although oligonucleotides may be double stranded, e.g., for use in the construction of a gene mutant. Oligonucleotides can be either sense or antisense oligonucleotides.

The term “naturally occurring nucleotide” as used herein includes deoxyribonucleotides and ribonucleotides. The term “modified nucleotides” referred to herein includes nucleotides with modified or substituted sugar groups and the like. The term “oligonucleotide linkages” referred to herein includes oligonucleotides linkages such as phosphorothioate, phosphorodithioate, phosphoroselenoate, phosphorodiselenoate, phosphoroanilothioate, phosphoraniladate, phosphoroamidate, and the like. See e.g., LaPlanche et al. Nucl. Acids Res. 14:9081 (1986); Stec et al. J. Am. Chem. Soc. 106:6077 (1984); Stein et al. Nucl. Acids Res. 16:3209 (1988); Zon et al. Anti-Cancer Drug Design 6:539 (1991); Zon et al. Oligonucleotides and Analogues. A Practical Approach, pp. 87-108 (F. Eckstein, Ed., Oxford University Press, Oxford England (1991)); Stec et al. U.S. Patent No. 5,151,510; Uhlmann and Peyman Chemical Reviews 90:543 (1990), the disclosures of which are hereby incorporated by reference. An oligonucleotide can include a label for detection, if desired.

The term “selectively hybridize” referred to herein means to detectably and specifically bind. Polynucleotides, oligonucleotides and fragments thereof selectively hybridize to nucleic acid strands under hybridization and wash conditions that minimize appreciable amounts of detectable binding to nonspecific nucleic acids. High stringency conditions can be used to achieve selective hybridization conditions as known in the art and discussed herein. Generally, the nucleic acid sequence homology between the polynucleotides, oligonucleotides, or antibody fragments and a nucleic acid sequence of interest will be at least 80%, and more typically with preferably increasing homologies of at least 85%, 90%, 95%, 99%, and 100%.

The term “control sequence” as used herein refers to polynucleotide sequences which are necessary to effect the expression and processing of coding sequences to which they are connected. The nature of such control sequences differs depending upon the host organism; in prokaryotes, such control sequences generally include promoter, ribosomal binding site, and transcription termination sequence; in eukaryotes, generally, such control sequences include promoters and transcription termination sequence. The term “control sequences” is intended to include, at a minimum, all components whose presence is essential for expression and processing, and can also include additional components whose presence is advantageous, for example, leader sequences and fusion partner sequences.

The term “operably linked” as used herein refers to positions of components so described that are in a relationship permitting them to function in their intended manner. For example, a control sequence “operably linked” to a coding sequence is connected in such a way that expression of the coding sequence is achieved under conditions compatible with the control sequences.

The term “isolated protein” referred to herein means a protein of cDNA, recombinant RNA, or synthetic origin or some combination thereof, which by virtue of its origin, or source of derivation, the “isolated protein” (1) is not associated with proteins found in nature, (2) is free of other proteins from the same source, e.g., free of murine proteins, (3) is expressed by a cell from a different species, or (4) does not occur in nature.

The term “polypeptide” is used herein as a generic term to refer to native protein, fragments, or analogs of a polypeptide sequence. Hence, native protein, fragments, and analogs are species of the polypeptide genus. Preferred polypeptides in accordance with the invention comprise the human heavy chain immunoglobulin molecules represented by SEQ ID NOs: 2, 6, 10, 14,18, 22, 26, 30, 34, and 38, for example, and the human kappa light chain immunoglobulin molecules represented by SEQ ID NOs: 4, 8, 12, 16, 20, 24, 28, 32, 36, and 40, for example, as well as antibody molecules formed by combinations comprising the heavy chain immunoglobulin molecules with light chain immunoglobulin molecules, such as the kappa light chain immunoglobulin molecules, and vice versa, as well as fragments and analogs thereof.

Unless specified otherwise, the left-hand end of single-stranded polynucleotide sequences is the 5′ end; the left-hand direction of double-stranded polynucleotide sequences is referred to as the 5′ direction. The direction of 5′ to 3′ addition of nascent RNA transcripts is referred to as the transcription direction; sequence regions on the DNA strand having the same sequence as the RNA and which are 5′ to the 5′ end of the RNA transcript are referred to as “upstream sequences”; sequence regions on the DNA strand having the same sequence as the RNA and which are 3′ to the 3′ end of the RNA transcript are referred to as “downstream sequences”.

As used herein, the twenty conventional amino acids and their abbreviations follow conventional usage. See Immunology—A Synthesis (2nd Edition, E. S. Golub and D. R. Gren, Eds., Sinauer Associates, Sunderland, Mass. (1991)), which is incorporated herein by reference. Stereoisomers (e.g., D-amino acids) of the twenty conventional amino acids, unnatural amino acids such as alpha-, alpha-disubstituted amino acids, N-alkyl amino acids, lactic acid, and other unconventional amino acids may also be suitable components for polypeptides of the present invention. Examples of unconventional amino acids include: 4-hydroxyproline, γ-carboxyglutamate,ε-N,N,N-trimethyllysine, ε-N-acetyllysine, O-phosphoserine, N-acetylserine, N-formylmethionine, 3-methylhistidine, 5-hydroxylysine, σ-N-methylarginine, and other similar amino acids and imino acids (e.g., 4-hydroxyproline). In the polypeptide notation used herein, the lefthand direction is the amino terminal direction and the righthand direction is the carboxy-terminal direction, in accordance with standard usage and convention.

The term “corresponds to” is used herein to mean that a polynucleotide sequence is homologous (i.e., is identical, not strictly evolutionarily related) to all or a portion of a reference polynucleotide sequence, or that a polypeptide sequence is identical to a reference polypeptide sequence.

In contradistinction, the term “complementary to” is used herein to mean that the complementary sequence is homologous to all or a portion of a reference polynucleotide sequence. For illustration, the nucleotide sequence “TATAC” corresponds to a reference sequence “TATAC” and is complementary to a reference sequence “GTATA”.

The following terms are among those used to describe the sequence relationships between two or more polynucleotide or amino acid sequences: “reference sequence”, “comparison window”, “sequence identity”, “percentage of sequence identity”, “substantial identity”, and “homology.” A “reference sequence” is a defined sequence used as a basis for a sequence comparison. A reference sequence may be a subset of a larger sequence, for example, as a segment of a full-length cDNA or gene sequence given in a sequence listing or may comprise a complete cDNA or gene sequence. Generally, a reference sequence is at least 18 nucleotides or 6 amino acids in length, frequently at least 24 nucleotides or 8 amino acids in length, and often at least 48 nucleotides or 16 amino acids in length. Since two polynucleotides or amino acid sequences may each (1) comprise a sequence (i.e., a portion of the complete polynucleotide or amino acid sequence) that is similar between the two molecules, and (2) may further comprise a sequence that is divergent between the two polynucleotides or amino acid sequences, sequence comparisons between two (or more) molecules are typically performed by comparing sequences of the two molecules over a “comparison window” to identify and compare local regions of sequence similarity.

A “comparison window”, as used herein, refers to a conceptual segment of at least about 18 contiguous nucleotide positions or about 6 amino acids wherein the polynucleotide sequence or amino acid sequence is compared to a reference sequence of at least 18 contiguous nucleotides or 6 amino acid sequences and wherein the portion of the polynucleotide sequence in the comparison window may include additions, deletions, substitutions, and the like (i.e., gaps) of 20 percent or less as compared to the reference sequence (which does not comprise additions or deletions) for optimal alignment of the two sequences. Optimal alignment of sequences for aligning a comparison window may be conducted by the local homology algorithm of Smith and Waterman Adv. Appl. Math. 2:482 (1981), by the homology alignment algorithm of Needleman and Wunsch J. Mol. Biol. 48:443 (1970), by the search for similarity method of Pearson and Lipman Proc. Natl. Acad. Sci. (U.S.A.) 85:2444 (1988), by computerized implementations of these algorithms (GAP, BESTFIT, FASTA, and TFASTA in the Wisconsin Genetics Software Package Release 7.0, (Genetics Computer Group, 575 Science Dr., Madison, Wis.), GENEWORKS™, or MACVECTOR® software packages), or by inspection, and the best alignment (i.e., resulting in the highest percentage of homology over the comparison window) generated by the various methods is selected.

The term “sequence identity” means that two polynucleotide or amino acid sequences are identical (i.e., on a nucleotide-by-nucleotide or residue-by-residue basis) over the comparison window. The term “percentage of sequence identity” is calculated by comparing two optimally aligned sequences over the window of comparison, determining the number of positions at which the identical nucleic acid base (e.g., A, T, C, G, U, or I) or amino acid residue occurs in both sequences to yield the number of matched positions, dividing the number of matched positions by the total number of positions in the comparison window (i.e., the window size), and multiplying the result by 100 to yield the percentage of sequence identity. The terms “substantial identity” as used herein denotes a characteristic of a polynucleotide or amino acid sequence, wherein the polynucleotide or amino acid comprises a sequence that has at least 85 percent sequence identity, preferably at least 90 to 95 percent sequence identity, more preferably at least 99 percent sequence identity, as compared to a reference sequence over a comparison window of at least 18 nucleotide (6 amino acid) positions, frequently over a window of at least 24-48 nucleotide (8-16 amino acid) positions, wherein the percentage of sequence identity is calculated by comparing the reference sequence to the sequence which may include deletions or additions which total 20 percent or less of the reference sequence over the comparison window. The reference sequence may be a subset of a larger sequence.

Two amino acid sequences or polynucleotide sequences are “homologous” if there is a partial or complete identity between their sequences. For example, 85% homology means that 85% of the amino acids are identical when the two sequences are aligned for maximum matching. Gaps (in either of the two sequences being matched) are allowed in maximizing matching; gap lengths of 5 or less are preferred with 2 or less being more preferred. Alternatively and preferably, two protein sequences (or polypeptide sequences derived from them of at least about 30 amino acids in length) are homologous, as this term is used herein, if they have an alignment score of at more than 5 (in standard deviation units) using the program ALIGN with the mutation data matrix and a gap penalty of 6 or greater. See Dayhoff, M.O., in Atlas of Protein Sequence and Structure, pp. 101-110 (Volume 5, National Biomedical Research Foundation (1972)) and Supplement 2 to this volume, pp. 1-10. The two sequences or parts thereof are more preferably homologous if their amino acids are greater than or equal to 50% identical when optimally aligned using the ALIGN program.

As applied to polypeptides, the term “substantial identity” means that two peptide sequences, when optimally aligned, such as by the programs GAP or BESTFIT using default gap weights, share at least 80 percent sequence identity, preferably at least 90 percent sequence identity, more preferably at least 95 percent sequence identity, and most preferably at least 99 percent sequence identity. Preferably, residue positions which are not identical differ by conservative amino acid substitutions. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, glutamic-aspartic, and asparagine-glutamine.

As discussed herein, minor variations in the amino acid sequences of antibodies or immunoglobulin molecules are contemplated as being encompassed by the present invention, providing that the variations in the amino acid sequence maintain at least 75%, more preferably at least 80%, 90%, 95%, and most preferably 99%. In particular, conservative amino acid replacements are contemplated. Conservative replacements are those that take place within a family of amino acids that are related in their side chains. Genetically encoded amino acids are generally divided into families: (1) acidic=aspartate, glutamate; (2) basic=lysine, arginine, histidine; (3) non-polar=alanine, valine, leucine, isoleucine, proline, phenylalanine, methionine, tryptophan; and (4) uncharged polar=glycine, asparagine, glutamine, cysteine, serine, threonine, tyrosine. More preferred families are: serine and threonine are aliphatic-hydroxy family; asparagine and glutamine are an amide-containing family; alanine, valine, leucine and isoleucine are an aliphatic family; and phenylalanine, tryptophan, and tyrosine are an aromatic family. For example, it is reasonable to expect that an isolated replacement of a leucine with an isoleucine or valine, an aspartate with a glutamate, a threonine with a serine, or a similar replacement of an amino acid with a structurally related amino acid will not have a major effect on the binding or properties of the resulting molecule, especially if the replacement does not involve an amino acid within a framework site. Whether an amino acid change results in a functional peptide can readily be determined by assaying the specific activity of the polypeptide derivative. Assays are described in detail herein. Fragments or analogs of antibodies or immunoglobulin molecules can be readily prepared by those of ordinary skill in the art. Preferred amino- and carboxy-termini of fragments or analogs occur near boundaries of functional domains. Structural and functional domains can be identified by comparison of the nucleotide and/or amino acid sequence data to public or proprietary sequence databases. Preferably, computerized comparison methods are used to identify sequence motifs or predicted protein conformation domains that occur in other proteins of known structure and/or function. Methods to identify protein sequences that fold into a known three-dimensional structure are known. Bowie et al. Science 253:164 (1991). The foregoing examples demonstrate that those of skill in the art can recognize sequence motifs and structural conformations that may be used to define structural and functional domains in accordance with the invention.

Preferred amino acid substitutions are those which: (1) reduce susceptibility to proteolysis, (2) reduce susceptibility to oxidation, (3) alter binding affinity for forming protein complexes, (4) alter binding affinities, and (5) confer or modify other physiocochemical or functional properties of such analogs. Analogs can include various muteins of a sequence other than the naturally-occurring peptide sequence. For example, single or multiple amino acid substitutions (preferably conservative amino acid substitutions) may be made in the naturally-occurring sequence (preferably in the portion of the polypeptide outside the domain(s) forming intermolecular contacts. A conservative amino acid substitution should not substantially change the structural characteristics of the parent sequence (e.g., a replacement amino acid should not tend to break a helix that occurs in the parent sequence, or disrupt other types of secondary structure that characterizes the parent sequence). Examples of art-recognized polypeptide secondary and tertiary structures are described in Proteins, Structures and Molecular Principles (Creighton, Ed., W. H. Freeman and Company, New York (1984)); Introduction to Protein Structure (C. Branden and J. Tooze, eds., Garland Publishing, New York, N.Y. (1991)); and Thornton et at. Nature 354:105 (1991), which are each incorporated herein by reference.

The term “polypeptide fragment” as used herein refers to a polypeptide that has an amino-terminal and/or carboxy-terminal deletion, but where the remaining amino acid sequence is identical to the corresponding positions in the naturally-occurring sequence deduced, for example, from a full-length cDNA sequence. Fragments typically are at least 5, 6, 8 or 10 amino acids long, preferably at least 14 amino acids long, more preferably at least 20 amino acids long. In other embodiments polypeptide fragments are at least 25 amino acids long, more preferably at least 50 amino acids long, and even more preferably at least 70 amino acids long.

Peptide analogs are commonly used in the pharmaceutical industry as non-peptide drugs with properties analogous to those of the template peptide. These types of non-peptide compound are termed “peptide mimetics” or “peptidomimetics”. Fauchere, J. Adv. Drug Res. 15:29 (1986); Veber and Freidinger TINS p.392 (1985); and Evans et al. J. Med. Chem. 30:1229 (1987), which are incorporated herein by reference. Such compounds are often developed with the aid of computerized molecular modeling. Peptide mimetics that are structurally similar to therapeutically useful peptides may be used to produce an equivalent therapeutic or prophylactic effect. Generally, peptidomimetics are structurally similar to a paradigm polypeptide (i.e., a polypeptide that has a biochemical property or pharmacological activity), such as human antibody, but have one or more peptide linkages optionally replaced by a linkage selected from the group consisting of: —CH₂NH—, —CH₂S—, —CH₂—CH₂—, —CH═CH—(cis and trans), —COCH₂—, —CH(OH)CH₂—, and —CH₂SO—, by methods well known in the art. Systematic substitution of one or more amino acids of a consensus sequence with a D-amino acid of the same type (e.g., D-lysine in place of L-lysine) may be used to generate more stable peptides. In addition, constrained peptides comprising a consensus sequence or a substantially identical consensus sequence variation may be generated by methods known in the art (Rizo and Gierasch Ann. Rev. Biochem. 61:387 (1992), incorporated herein by reference); for example, by adding internal cysteine residues capable of forming intramolecular disulfide bridges which cyclize the peptide.

As used herein, the terms “label” or “labeled” refers to incorporation of a detectable marker, e.g., by incorporation of a radiolabeled amino acid or attachment to a polypeptide of biotinyl moieties that can be detected by marked avidin (e.g., streptavidin containing a fluorescent marker or enzymatic activity that can be detected by optical or colorimetric methods). In certain situations, the label or marker can also be therapeutic. Various methods of labeling polypeptides and glycoproteins are known in the art and may be used. Examples of labels for polypeptides include, but are not limited to, the following: radioisotopes or radionuclides (e.g., ³H, ¹⁴C, ¹⁵N, ³⁵S, ⁹⁰Y, ⁹⁹Tc, ¹¹¹In, ¹²⁵I, ¹³¹I), fluorescent labels (e.g., FITC, rhodamine, lanthanide phosphors), enzymatic labels (e.g., horseradish peroxidase, β-galactosidase, luciferase, alkaline phosphatase), chemiluminescent, biotinyl groups, predetermined polypeptide epitopes recognized by a secondary reporter (e.g., leucine zipper pair sequences, binding sites for secondary antibodies, metal binding domains, epitope tags). In some embodiments, labels are attached by spacer arms of various lengths to reduce potential steric hindrance.

The term “pharmaceutical agent or drug” as used herein refers to a chemical compound or composition capable of inducing a desired therapeutic effect when properly administered to a patient. Other chemistry terms herein are used according to conventional usage in the art, as exemplified by The McGraw-Hill Dictionary of Chemical Terms (Parker, S., Ed., McGraw-Hill, San Francisco (1985)), incorporated herein by reference).

An “agent” refers to a substance that is useful in the treatment of active disease, prophylactic treatment, or diagnosis of a mammal including, but not restricted to, a human, bovine, equine, porcine, murine, canine, feline, or any other warm-blooded animal. For example, the agent is selected from the group of radioisotope, toxin, pharmaceutical agent, oligonucleotide, cytotoxic agents, recombinant protein, antibody fragment, anti-cancer agents, anti-adhesion agents, anti-thrombosis agents, anti-restenosis agents, anti-autoimmune agents, anti-aggregation agents, anti-bacterial agents, anti-viral agents, and anti-inflammatory agents. Other examples of such agents include, but are not limited to anti-viral agents including acyclovir, ganciclovir, and zidovudine; anti-thrombosis/resteno- sis agents including cilostazol, dalteparin sodium, reviparin sodium, and aspirin; anti-inflammatory agents including zaltoprofen, pranoprofen, droxicam, acetyl salicylic 17, diclofenac, ibuprofen, dexibuprofen, sulindac, naproxen, amtolmetin, celecoxib, indomethacin, rofecoxib, and nimesulid; anti-autoimmune agents including leflunomide, denileukin diftitox, subreum, WinRho SDF, defibrotide, and cyclophosphamide; and anti-adhesion/anti-aggregation agents including limaprost, clorcromene, and hyaluronic acid. The term “agent” is meant to encompass any of the compounds known to one of skill in the art or disclosed herein that can influence a target cell or target area in a desired way. Agents can include labels and various therapeutics as well.

As used herein, “substantially pure” means an object species is the predominant species present (i.e., on a molar basis it is more abundant than any other individual species in the composition), and preferably a substantially purified fraction is a composition wherein the object species comprises at least about 50 percent (on a molar basis) of all macromolecular species present. Generally, a substantially pure composition will comprise more than about 80 percent of all macromolecular species present in the composition, more preferably more than about 85%, 90%, 95%, and 99%. Most preferably, the object species is purified to essential homogeneity (contaminant species cannot be detected in the composition by conventional detection methods) wherein the composition consists essentially of a single macromolecular species.

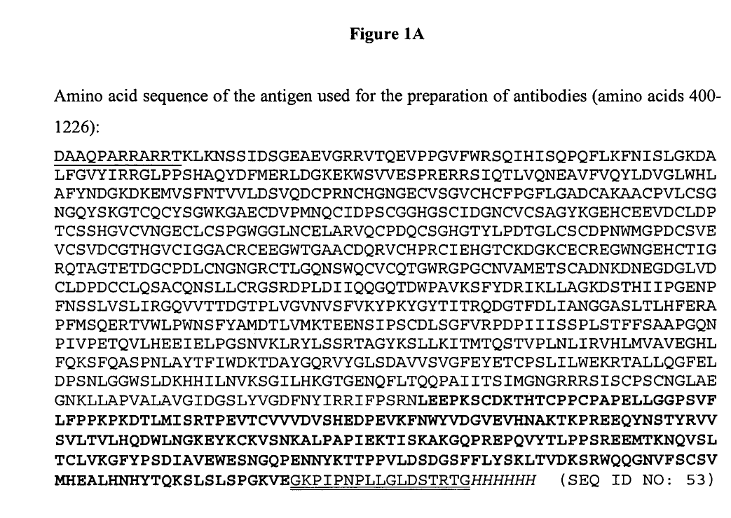

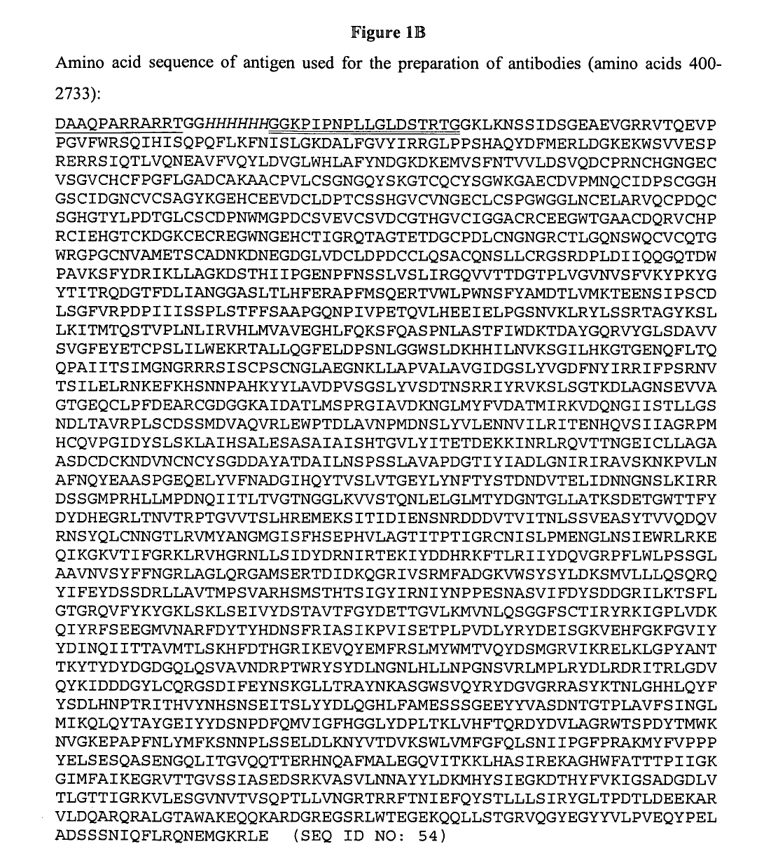

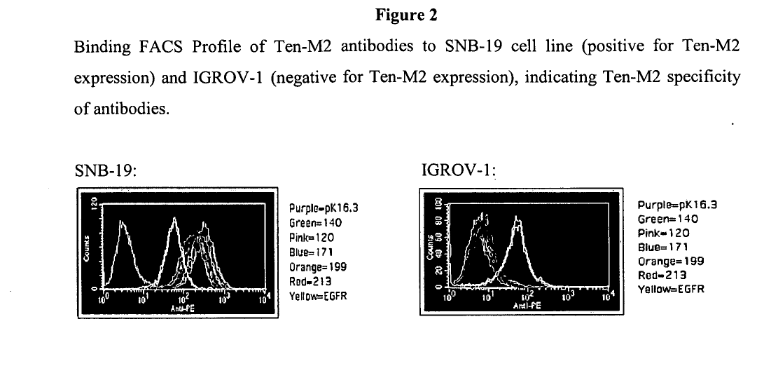

The term “Ten-M protein,” denotes a protein from the Ten-M family of genes, also known as teneurins, or hOdz. The Ten-M proteins are a class of type II transmembrane proteins containing a short intracellular N-terminus, which is followed by a transmembrane region, which is followed by 8 EGF-like repeats (Epidermal Growth Factor-like repeats), which is followed by a large globular domain on the extracellular side. Ten-M2 denotes a particular member of that family of proteins. Ten-M2 is also known as CG50426. The isolated sections of the Ten-M2 protein used in the preparation of the disclosed antibodies are shown in FIG. 1A, SEQ ID NO: 53 (amino acids 400-1226) and FIG. 1B, SEQ ID NO: 54 (amino acids 400-2733). As will be appreciated by one of skill in the art, and as described in more detail below, other sections of the Ten-M2 protein can be used to generate antibodies in the same manner as described herein.

Additionally, as will be appreciated by one of skill in the art, the present written description and enabling disclosure can readily be applied to create and use antibodies directed to the various members of the Ten-M family; however, for simplicity's sake, the Ten-M2 protein will be discussed as the example herein. Members of the Ten-M protein family have been described in rat (Otaki et al., Dev. Biol. 212, 165-1813 (1999)); chicken (Minet et al., J. Cell Sci. 112, 2019-2032 (1999); Rubin et al., R., Dev. Biol. 216, 195-209 (1999); Tucker et al., Mech. Dev. 98, 187-191 (2000); Tucker et al., Dev. Dyn. 220, 27-39 (2001)), human (Brandau et al., Hum. Mol. Genet. 8, 2407-2413 (1999); Minet et al., Gene 257, 87-97 (2000)), zebrafish (Mieda et al., Mech. Dev. 87, 223-227 (1999)), and Caenorhabditis elegans (Wilson et al., Nature 368, 32-38 (1994)).

Ten-M2 Antibodies

In some embodiments, the present antibodies can prevent the formation of a Ten-M2/Ten-M2 duplex on a cancerous cell and thereby reduce the likelihood that a cancer will spread to another location. As will be appreciated by one of skill in the art, the antibody can prevent or reduce the formation of the Ten-M2/Ten-M2 duplex formation in a number of ways. For example, the antibody can directly bind to the section of the Ten-M2 protein that is involved in binding for Ten-M2/Ten-M2 duplex formation (e.g., an EGF-like repeat) and thus prevent the two Ten-M2 proteins from effectively binding together. In some embodiments, the antibody directly binds to the EGF-like repeat or repeats that are involved in duplex formation. The antibody can be created to bind to any one or multiple EGF-like repeats, including, for example, the 1^{st}, 2^{nd}, 3^{rd}, 4^{th}, 5^{th}, 6^{th}, 7^{th}, and 8^{th}repeat. Thus, in one embodiment, the antibody can bind to the second and fourth EGF-like repeats. Fully human antibodies that bind to these particular sections can be generated by the methods disclosed herein and the knowledge of one of skill in the art. Alternatively, the antibody can bind at another location and the nonbonding section of the antibody can sterically interfere with the binding of the two halves of the protein. Alternatively, the antibody can bind to a location on the Ten-M2 proteins and induce a conformational change in the protein that will prevent duplex formation.

In some embodiments, the antibody binds in such a way as to allow for the binding of the two Ten-M2 proteins, but so as to prevent any functional signaling from occurring. In some embodiments, this involves the antibody binding to only a part of the section of the Ten-M2 protein directly involved in duplex formation (e.g., one EGF-like repeat), while the antibody does not interfere with the binding of the two Ten-M2 proteins otherwise (e.g., the other EGF-like repeat will still bind together). This particular antibody has the advantage of reducing the number of Ten-M2 proteins available to form duplexes, as each antibody can bind to two Ten-M2 proteins. Similarly, antibodies that bind to two Ten-M2 proteins at once can also have this advantage in some embodiments.

In some embodiments, the antibody actually promotes the dissociation of the Ten-M2/Ten-M2 duplex. In other embodiments, the antibody promotes the formation of the Ten-M2/Ten-M2 duplex. Such antibodies can be created by raising antibodies against particular locations of the Ten-M2 protein or duplex thereof, so that the antibody, when bound to the Ten-M2 protein or duplex thereof, will help stabilize the other state (individual or duplexed) of the Ten-M2 protein.

One of skill in the art will appreciate that with the combination of 1) the present teachings, 2) logical selection of the antigen from the Ten-M2 protein (e.g., one, some or all of the EGF-like repeats), 3) a standard antibody binding assay (e.g., surface plasmon resonance in a BIACORE™ device), and 4) a functional duplex formation assay (e.g., a cell migration assay similar to that described below), that the above antibodies can be readily generated identified, isolated and used.

In some embodiments, the antibodies to Ten-M2 are selective for various forms of Ten-M and various forms of Ten-M2. For example, in some embodiments, the antibody to Ten-M2 will bind to Ten-M2 more tightly than it will to other forms of Ten-M (e.g., Ten-M4, Ten-Ml, and Ten-M3). For example, the antibody can bind 1-5, 5-10, 10-20, 20-30, 30-40, or 40-50 fold more tightly to Ten-M2 than one of or any combination of the other Ten-M proteins. In other embodiments, the antibodies are selective for cell associated and cell dissociated Ten-M proteins and Ten-M2 in particular. For example, in some embodiments, the antibodies can bind more tightly to a Ten-M2 protein that is attached to the cell surface. In other embodiments, the antibodies can bind more tightly to a Ten-M2 protein that has been shed or that is secreted or cleaved from a cell. In some embodiments, the antibody can bind to both forms equally as strongly. In other embodiments, the antibody can effectively bind to only one form of the Ten-M2 protein. The selectivity can be any amount, for example, from 2-10, 10-20, 20-30, 30-40, 40-50, fold more selective, or more, for one form compared to the other form. An example of how to generate such selective antibodies and determine such selectivity is presented below in the examples.

In other embodiments, the antibodies that bind to Ten-M2 are associated with an agent or compound of some type. The association of the agent with the antibody allows for the delivery of the agent or compound to cells that express Ten-M2. As observed, Ten-M2 is expressed in cancerous cells; therefore, this combination allows the delivery of an agent, such as a cytotoxic agent or therapeutic agent, to a cancer cell. The agent can be associated with the antibody in a variety of ways, for example, it can be directly linked to the antibody, attached via a linker (which can be a cleavable linker), or associated via a secondary antibody. In some embodiments the antibodies comprise epitopes so as to allow binding to the antibodies by other antibodies or agents. As will be appreciated by one of skill in the art, the exact manner by which the agent is associated with the toxin is not critical to the device or method. This, and other issues associated with these compositions and methods of using them are discussed in more detail below, with a particular emphasis in the section entitled “Design and Generation of Other Therapeutics.”

Antibody Structure

The basic antibody structural unit is known to comprise a tetramer. Each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. Human light chains are classified as kappa and lambda light chains. Heavy chains are classified as mu, delta, gamma, alpha, or epsilon, and define the antibody's isotype as IgM, IgD, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989)) (incorporated by reference in its entirety for all purposes). The variable regions of each light/heavy chain pair form the antibody binding site.

Thus, an intact antibody has two binding sites. Except in bifunctional or bispecific antibodies, the two binding sites are the same.

The chains all exhibit the same general structure of relatively conserved framework regions (FR) joined by three hyper variable regions, also called complementarity determining regions or CDRs. The CDRs from the two chains of each pair are aligned by the framework regions, enabling binding to a specific epitope. From N-terminal to C-terminal, both light and heavy chains comprise the domains FR1, CDR1, FR2, CDR2, FR3, CDR3 and FR4. The assignment of amino acids to each domain is in accordance with the definitions of Kabat Sequences of Proteins of Immunological Interest (National Institutes of Health, Bethesda, Md. (1987 and 1991)), or Chothia & Lesk J. Mol. Biol. 196:901-917 (1987); Chothia et al. Nature 342:878-883 (1989).

A bispecific or bifunctional antibody is an artificial hybrid antibody having two different heavy/light chain pairs and two different binding sites. Bispecific antibodies can be produced by a variety of methods including fusion of hybridomas or linking of Fab′ fragments. See, e.g., Songsivilai & Lachmann Clin. Exp. Immunol. 79: 315-321 (1990), Kostelny et al. J. Immunol. 148:1547-1553 (1992). Production of bispecific antibodies can be a relatively labor intensive process compared with production of conventional antibodies and yields and degree of purity are generally lower for bispecific antibodies. Bispecific antibodies do not exist in the form of fragments having a single binding site (e.g., Fab, Fab′, and Fv).

Human Antibodies and Humanization of Antibodies

Human antibodies avoid some of the problems associated with antibodies that possess murine or rat variable and/or constant regions. The presence of such murine or rat derived proteins can lead to the rapid clearance of the antibodies or can lead to the generation of an immune response against the antibody by a patient. In order to avoid the utilization of murine or rat derived antibodies, fully human antibodies can be generated through the introduction of human antibody function into a rodent so that the rodent produces fully human antibodies.

One method for generating fully human antibodies is through the use of XENOMOUSE® strains of mice which have been engineered to contain 245 kb and 190 kb-sized germline configuration fragments of the human heavy chain locus and kappa light chain locus. See Green et al. Nature Genetics 7:13-21 (1994). The XENOMOUSE® strains are available from Abgenix, Inc. (Fremont, Calif.).

The production of the XENOMOUSE® is further discussed and delineated in U.S. patent application Ser. Nos. 07/466,008, filed Jan. 12, 1990, Ser. No. 07/610,515, filed Nov. 8, 1990, Ser. No. 07/919,297, filed Jul. 24, 1992, Ser. No. 07/922,649, filed Jul. 30, 1992, Ser. No. 08/031,801, filed Mar. 15,1993, Ser. No. 08/112,848, filed August 27, 1993, 08/234,145, filed Apr. 28, 1994, Ser. No. 08/376,279, filed Jan. 20, 1995, Ser. No. 08/430, 938, April 27, 1995, 08/464,584, filed Jun. 5, 1995, Ser. No. 08/464,582, filed Jun. 5, 1995, Ser. No. 08/463,191, filed Jun. 5, 1995, Ser. No. 08/462,837, filed Jun. 5, 1995, Ser. No. 08/486,853, filed Jun. 5, 1995, Ser. No. 08/486,857, filed Jun. 5, 1995, Ser. No. 08/486,859, filed Jun. 5, 1995, Ser. No. 08/462,513, filed June 5, 1995, 08/724,752, filed Oct. 2, 1996, and Ser. No. 08/759,620, filed Dec. 3, 1996 and U.S. Pat. Nos. 6,162,963, 6,150,584, 6,114,598, 6,075,181, and 5,939,598 and Japanese Patent Nos. 3 068 180 B2, 3 068 506 B2, and 3 068 507 B2. See also Mendez et al. Nature Genetics 15:146-156 (1997) and Green and Jakobovits J. Exp. Med. 188:483-495 (1998). See also European Patent No., EP 0 463 151 B1, grant published Jun. 12, 1996, International Patent Application No., WO 94/02602, published Feb. 3, 1994, International Patent Application No., WO 96/34096, published Oct. 31, 1996, WO 98/24893, published Jun. 11, 1998, WO 00/76310, published Dec. 21, 2000. The disclosures of each of the above-cited patents, applications, and references are hereby incorporated by reference in their entirety.

In an alternative approach, others, including GenPharm International, Inc., have utilized a “minilocus” approach. In the minilocus approach, an exogenous Ig locus is mimicked through the inclusion of pieces (individual genes) from the Ig locus. Thus, one or more V_{H} genes, one or more D_{H} genes, one or more J_{H} genes, a mu constant region, and a second constant region (preferably a gamma constant region) are formed into a construct for insertion into an animal. This approach is described in U.S. Pat. No. 5,545,807 to Surani et al. and U.S. Pat. Nos. 5,545,806, 5,625,825, 5,625,126, 5,633,425, 5,661,016, 5,770,429, 5,789,650, 5,814,318, 5,877,397, 5,874,299, and 6,255,458 each to Lonberg and Kay, U.S. Pat. No. 5,591,669 and 6,023.010 to Krimpenfort and Berns, U.S. Pat. Nos. 5,612,205, 5,721,367, and 5,789,215 to Berns et al., and U.S. Pat. No. 5,643,763 to Choi and Dunn, and GenPharm International U.S. patent application Ser. No. 07/574,748, filed Aug. 29, 1990, Ser. No. 07/575,962, filed Aug. 31, 1990, Ser. No. 07/810,279, filed Dec. 17, 1991, Ser. No. 07/853,408, filed Mar. 18, 1992, Ser. No. 07/904,068, filed Jun. 23, 1992, Ser. No. 07/990,860, filed Dec. 16, 1992, Ser. No. 08/053,131, filed Apr. 26, 1993, Ser. No. 08/096,762, filed Jul. 22, 1993, Ser. No. 08/155,301, filed Nov. 18, 1993, Ser. No. 08/161,739, filed Dec. 3, 1993, 08/165,699, filed Dec. 10, 1993, Ser. No. 08/209,741, filed Mar. 9, 1994, the disclosures of which are hereby incorporated by reference. See also European Patent No. 0 546 073 B1, International Patent Application Nos. WO 92/03918, WO 92/22645, WO 92/22647, WO 92/22670, WO 93/12227, WO 94/00569, WO 94/25585, WO 96/14436, WO 97/13852, and WO 98/24884 and U.S. Pat. No. 5,981,175, the disclosures of which are hereby incorporated by reference in their entirety. See further Taylor et al., 1992, Chen et al., 1993, Tuaillon et al., 1993, Choi et al., 1993, Lonberg et al., (1994), Taylor et al., (1994), and Tuaillon et al., (1995), Fishwild et al., (1996), the disclosures of which are hereby incorporated by reference in their entirety.

Kirin has also demonstrated the generation of human antibodies from mice in which, through microcell fusion, large pieces of chromosomes, or entire chromosomes, have been introduced. See European Patent Application Nos. 773 288 and 843 961, the disclosures of which are hereby incorporated by reference in their entireties.

Human anti-mouse antibody (HAMA) responses have also led the industry to prepare chimeric or otherwise humanized antibodies. While chimeric antibodies have a human constant region and a murine variable region, it is expected that certain human anti-chimeric antibody (HACA) responses will be observed, particularly in chronic or multi-dose utilizations of the antibody. Thus, it would be desirable to provide fully human antibodies against multimeric enzymes in order to vitiate concerns and/or effects of HAMA or HACA response.

Preparation of Antibodies

Antibodies, as described herein, were prepared using the XENOMOUSE® technology, as described below. Such mice are capable of producing human immunoglobulin molecules and antibodies and are deficient in the production of murine immunoglobulin molecules and antibodies. Technologies utilized for achieving the same are disclosed in the patents, applications, and references referred to herein. In particular, however, a preferred embodiment of transgenic production of mice and antibodies therefrom is disclosed in U.S. patent application Ser. No. 08/759,620, filed Dec. 3, 1996 and International Patent Application Nos. WO 98/24893, published June 11, 1998 and WO 00/76310, published Dec. 21, 2000, the disclosures of which are hereby incorporated by reference. See also Mendez et al. Nature Genetics 15:146-156 (1997), the disclosure of which is hereby incorporated by reference.

Through use of such technology, fully human monoclonal antibodies to Ten-M2 have been produced, as described in detail below. Essentially, XENOMOUSE® lines of mice are immunized with an antigen of interest (e.g., human Ten-M2), lymphatic cells (such as B-cells) are recovered from mice that expressed antibodies, and the recovered cell lines are fused with a myeloid-type cell line to prepare immortal hybridoma cell lines. These hybridoma cell lines are screened and selected to identify hybridoma cell lines that produced antibodies specific to the antigen of interest. Provided herein are methods for the production of multiple hybridoma cell lines that produce antibodies specific to the desired multimeric enzyme subunit oligomerization domain. Further, provided herein are characterization of the antibodies produced by such cell lines, including nucleotide and amino acid sequence analyses of the heavy and light chains of such antibodies.

Alternatively, instead of being fused to myeloma cells to generate hybridomas, the recovered cells, isolated from immunized XENOMOUSE® lines of mice, are screened further for reactivity against the initial antigen, preferably human Ten-M2. Such screening includes ELISA with the desired Ten-M2 protein and functional assays such as Ten-M2 mediated antibody internalization. (Single B cells secreting antibodies of interest are then isolated using a desired Ten-M2-specific hemolytic plaque assay (Babcook et al., Proc. Natl. Acad. Sci. USA, i93:7843-7848 (1996)). Cells targeted for lysis are preferably sheep red blood cells (SRBCs) coated with the desired Ten-M2 antigen. In the presence of a B cell culture secreting the immunoglobulin of interest and complement, the formation of a plaque indicates specific Ten-M2-mediated lysis of the target cells.

The single antigen-specific plasma cell in the center of the plaque can be isolated and the genetic information that encodes the specificity of the antibody is isolated from the single plasma cell. Using reverse-transcriptase PCR, the DNA encoding the variable region of the antibody secreted can be cloned. Such cloned DNA can then be further inserted into a suitable expression vector, preferably a vector cassette such as a pcDNA, more preferably such a pcDNA vector containing the constant domains of immunoglobulin heavy and light chain. The generated vector can then be transfected into host cells, preferably CHO cells, and cultured in conventional nutrient media modified as appropriate for inducing promoters, selecting transformants, or amplifying the genes encoding the desired sequences. Herein, is described the isolation of multiple single plasma cells that produce antibodies specific to Ten-M2. Further, the genetic material that encodes the specificity of the anti-Ten-M2 antibody is isolated, and introduced into a suitable expression vector that is then transfected into host cells.

In general, antibodies produced by the above-mentioned cell lines possessed fully human IgG1 or IgG2 heavy chains with human kappa light chains. The antibodies possessed high affinities, typically possessing Kd's of from about 10-⁹ through about 10-13 M, when measured by either solid phase and solution phase.

As mentioned above, anti-Ten-M2 antibodies can be expressed in cell lines other than hybridoma cell lines. Sequences encoding particular antibodies can be used for transformation of a suitable mammalian host cell, such as a CHO cell. Transformation can be by any known method for introducing polynucleotides into a host cell, including, for example packaging the polynucleotide in a virus (or into a viral vector) and transducing a host cell with the virus (or vector) or by transfection procedures known in the art, as exemplified by U.S. Pat. Nos. 4,399,216, 4,912,040, 4,740,461, and 4,959,455 (which patents are hereby incorporated herein by reference). The transformation procedure used depends upon the host to be transformed. Methods for introducing heterologous polynucleotides into mammalian cells are well known in the art and include dextran-mediated transfection, calcium phosphate precipitation, polybrene mediated transfection, protoplast fusion, electroporation, encapsulation of the polynucleotide(s) in liposomes, and direct microinjection of the DNA into nuclei.

Mammalian cell lines available as hosts for expression are well known in the art and include many immortalized cell lines available from the American Type Culture Collection (ATCC), including but not limited to Chinese hamster ovary (CHO) cells, HeLa cells, baby hamster kidney (BHK) cells, monkey kidney cells (COS), human hepatocellular carcinoma cells (e.g., Hep G2), and a number of other cell lines. Cell lines of particular preference are selected through determining which cell lines have high expression levels and produce antibodies with Ten-M2 binding properties.

As will be appreciated by one of skill in the art, simply the selection of an antigen does not automatically mean that antibodies generated from the antigen will bind to the full protein in its native environment. Thus, in some embodiments, antibodies are tested for and selected for binding to the native protein, rather than variants of the original protein or antigen. In some embodiments, it is these proteins that are specifically contemplated.

Antibody Sequences

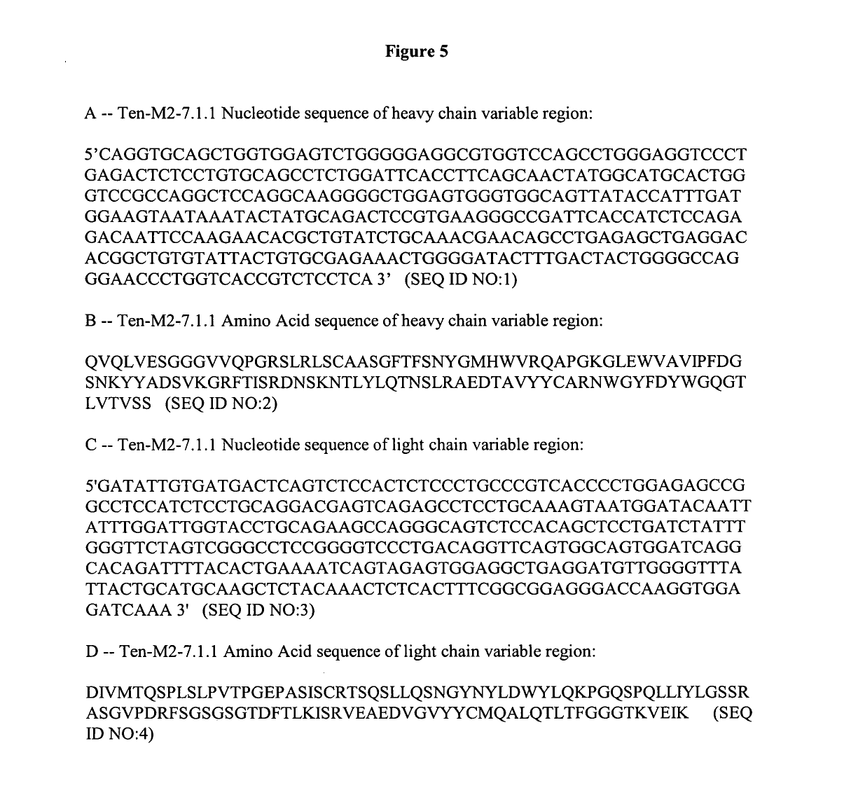

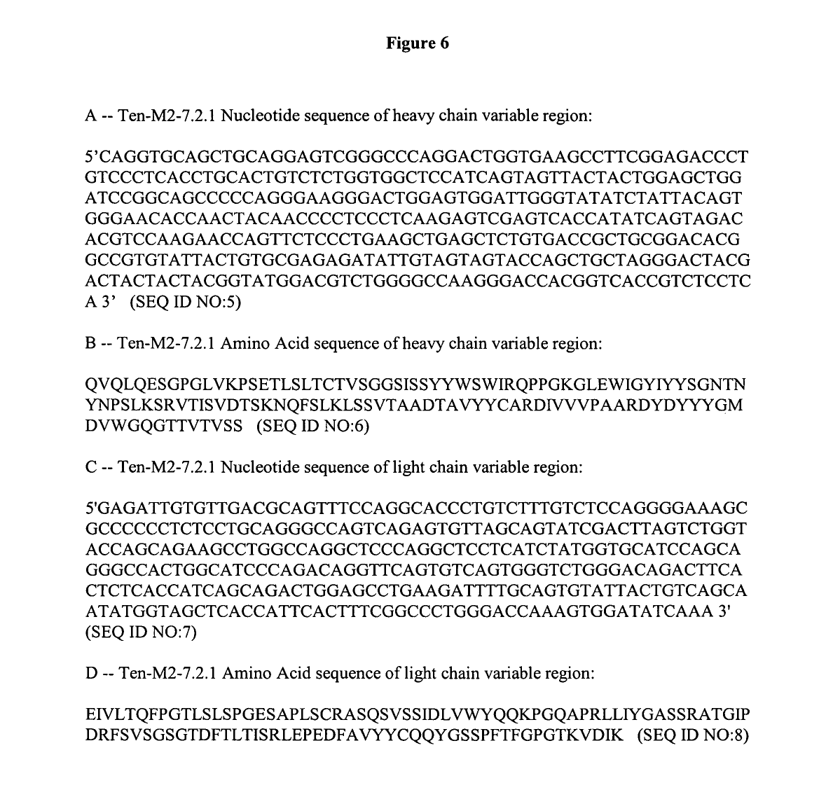

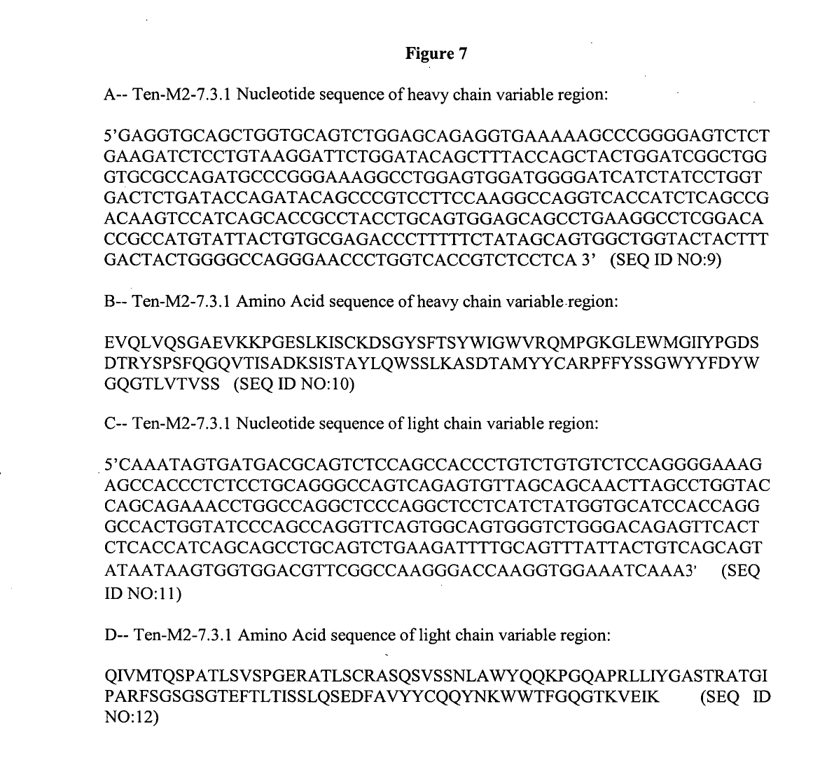

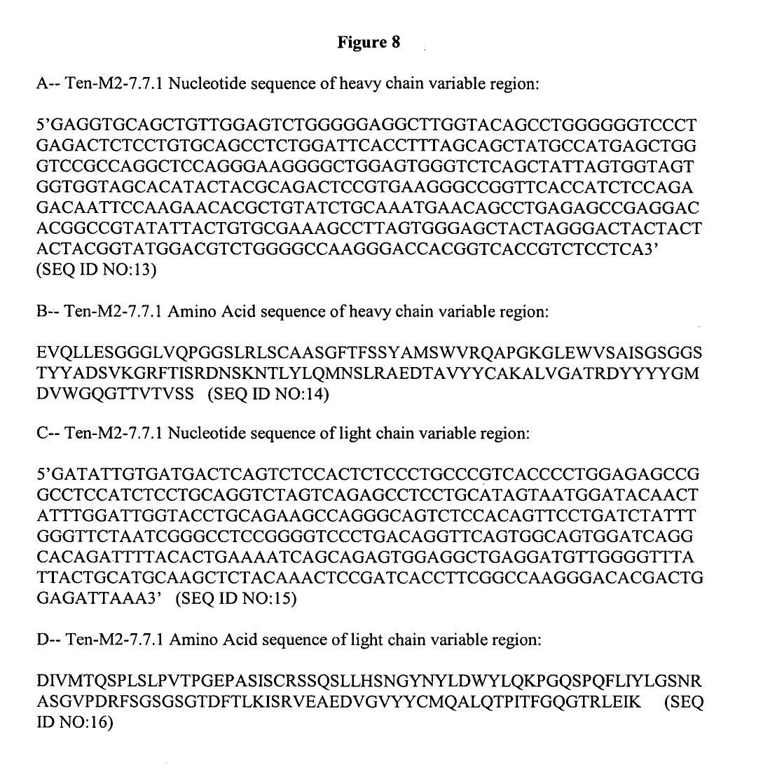

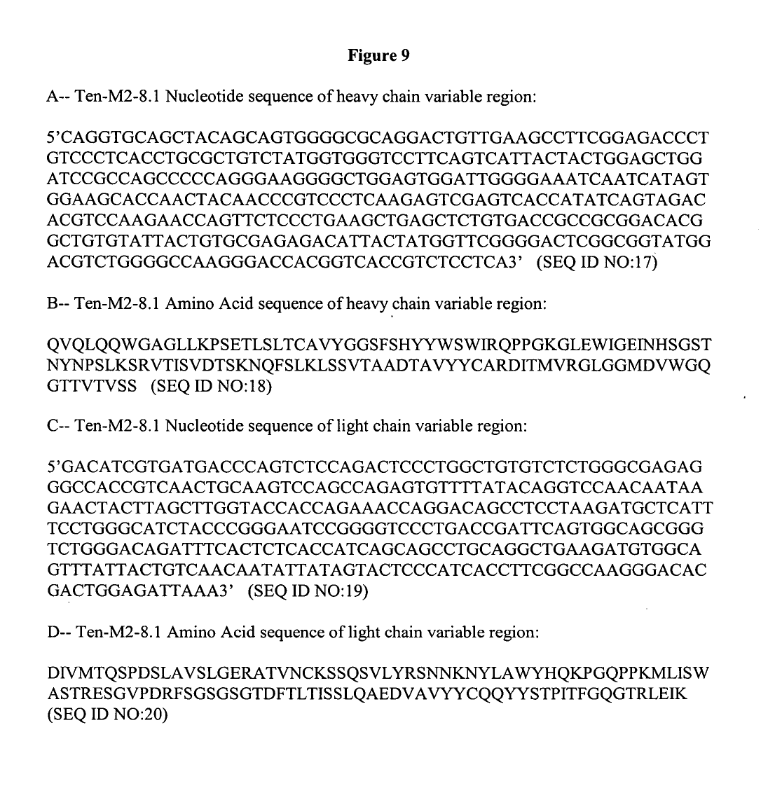

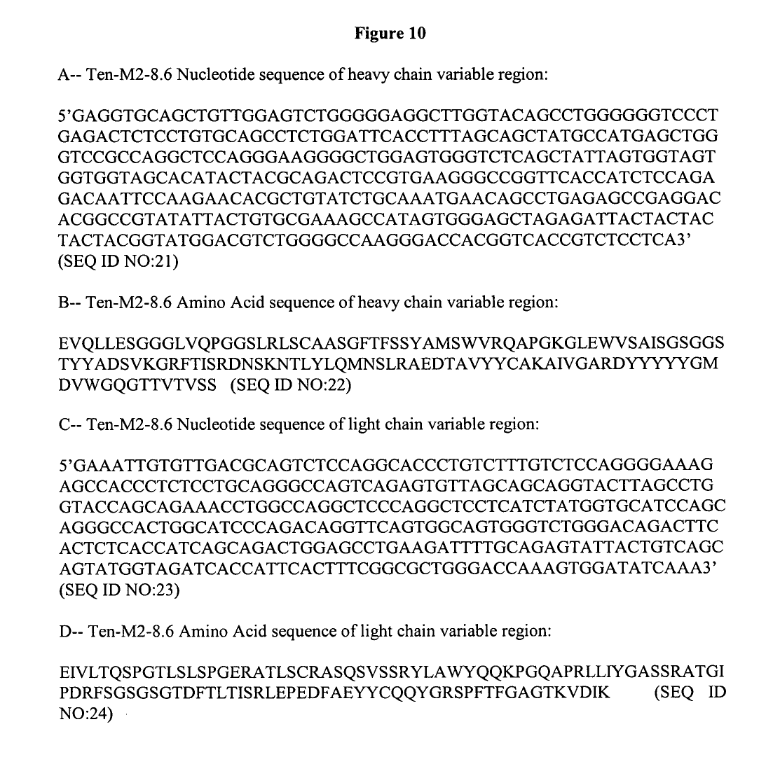

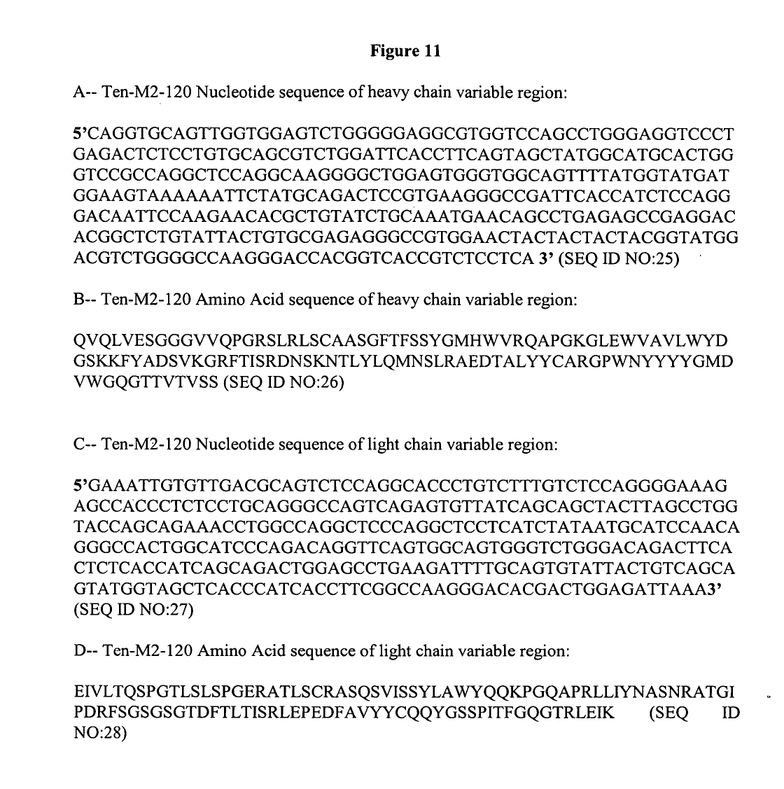

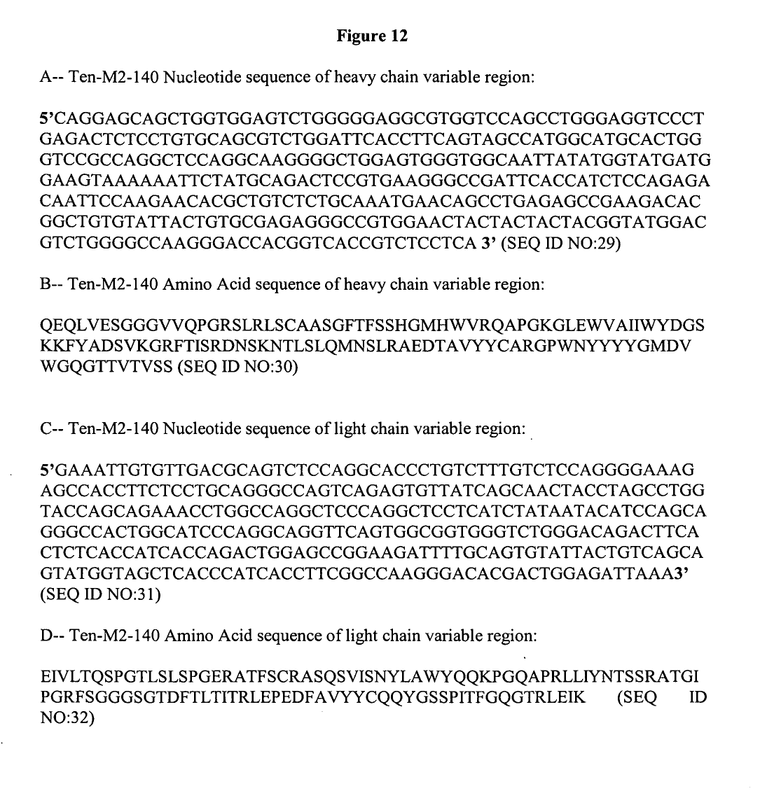

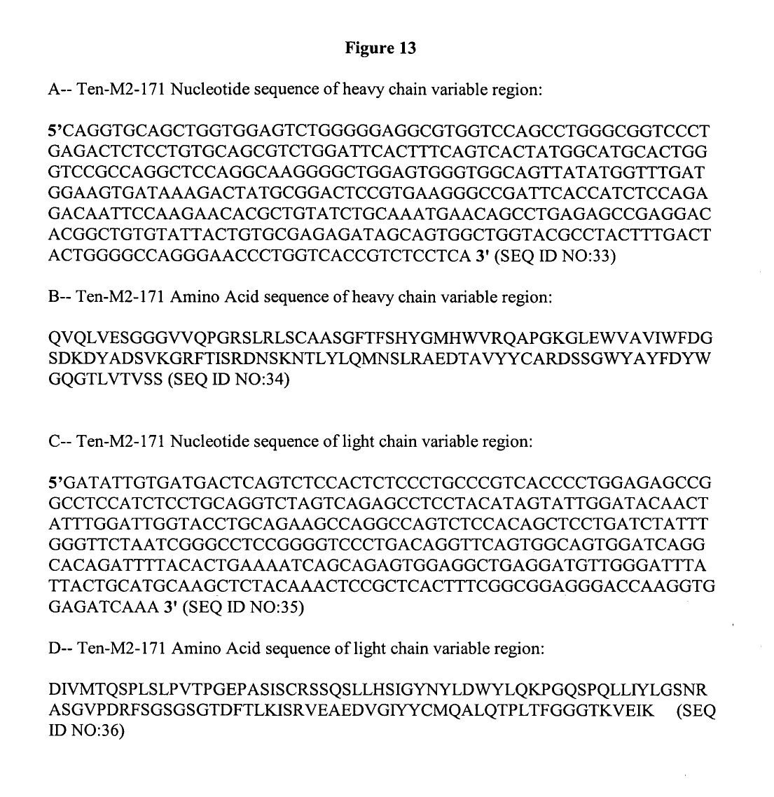

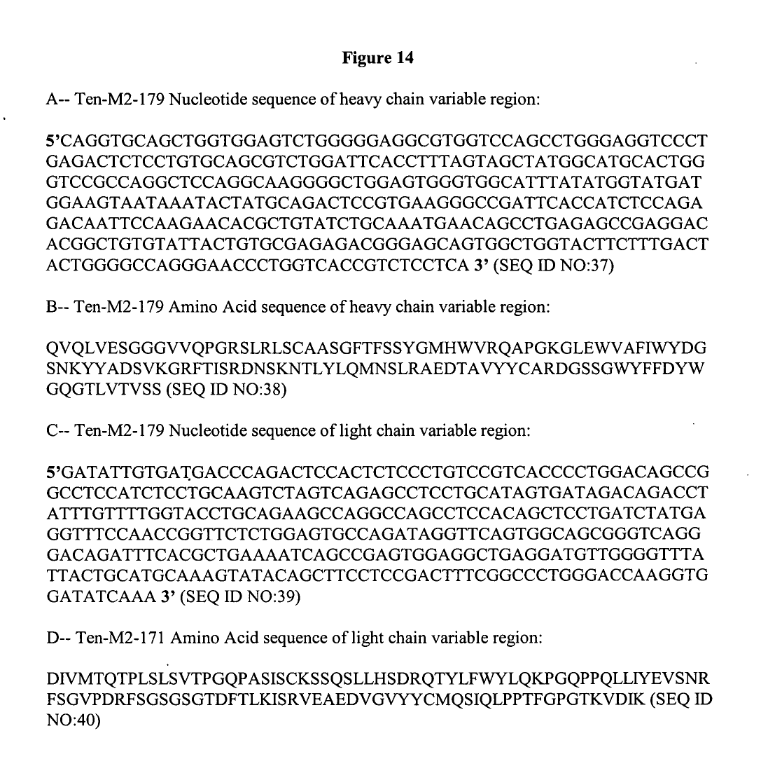

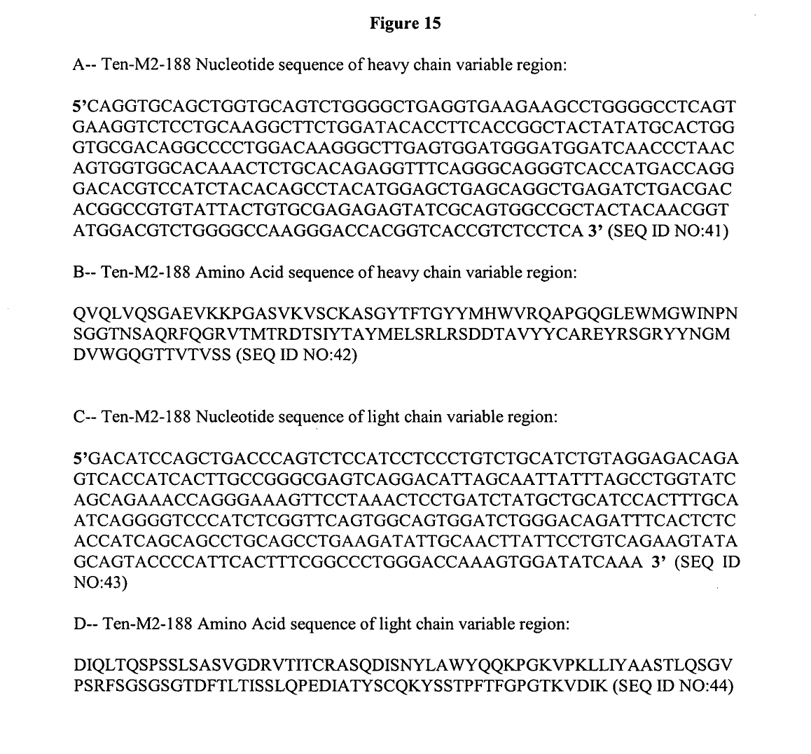

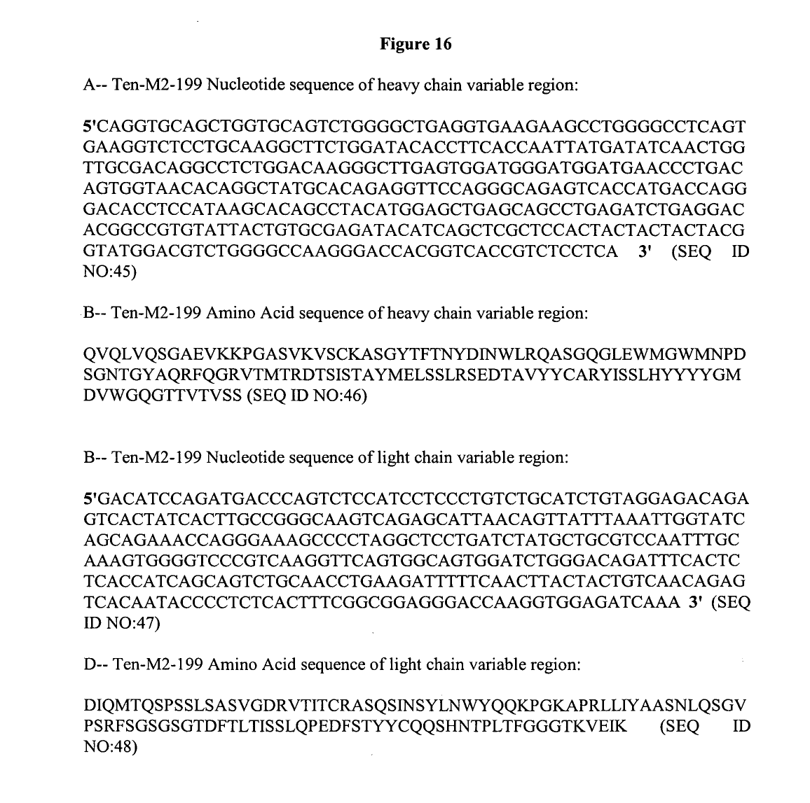

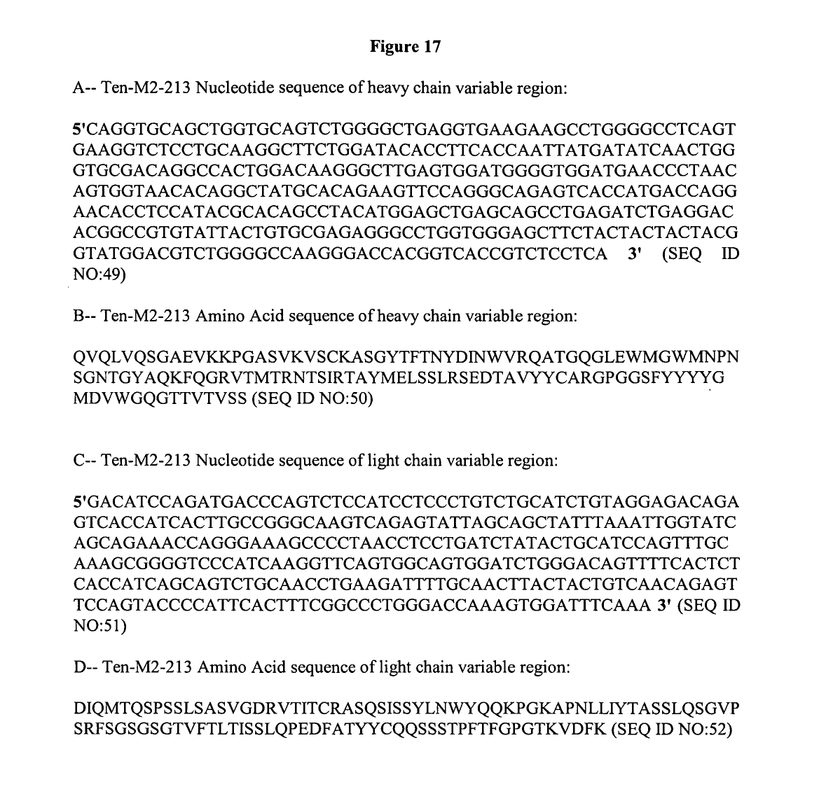

The heavy chain and light chain variable region nucleotide and amino acid sequences of representative human anti-Ten-M2 antibodies are provided in the sequence listing, the contents of which are summarized in Table 1 below and in FIGS. 5 through 17.

TABLE 1

| mAb | | SEQ ID |
|---|---|---|
| ID No.: | Sequence | NO: |
| 7.1.1 | Nucleotide sequence encoding the variable region of the heavy chain | 1 |
| | Amino acid sequence encoding the variable region of the heavy chain | 2 |
| | Nucleotide sequence encoding the variable region of the light chain | 3 |
| | Amino acid sequence encoding the variable region of the light chain | 4 |
| 7.2.1 | Nucleotide sequence encoding the variable region of the heavy chain | 5 |
| | Amino acid sequence encoding the variable region of the heavy chain | 6 |
| | Nucleotide sequence encoding the variable region of the light chain | 7 |
| | Amino acid sequence encoding the variable region of the light chain | 8 |
| 7.3.1 | Nucleotide sequence encoding the variable region of the heavy chain | 9 |
| | Amino acid sequence encoding the variable region of the heavy chain | 10 |
| | Nucleotide sequence encoding the variable region of the light chain | 11 |
| | Amino acid sequence encoding the variable region of the light chain | 12 |
| 7.7.1 | Nucleotide sequence encoding the variable region of the heavy chain | 13 |
| | Amino acid sequence encoding the variable region of the heavy chain | 14 |
| | Nucleotide sequence encoding the variable region of the light chain | 15 |
| | Amino acid sequence encoding the variable region of the light chain | 16 |
| 8.1 | Nucleotide sequence encoding the variable region of the heavy chain | 17 |
| | Amino acid sequence encoding the variable region of the heavy chain | 18 |
| | Nucleotide sequence encoding the variable region of the light chain | 19 |
| | Amino acid sequence encoding the variable region of the light chain | 20 |
| 8.6 | Nucleotide sequence encoding the variable region of the heavy chain | 21 |
| | Amino acid sequence encoding the variable region of the heavy chain | 22 |
| | Nucleotide sequence encoding the variable region of the light chain | 23 |
| | Amino acid sequence encoding the variable region of the light chain | 24 |
| 120 | Nucleotide sequence encoding the variable region of the heavy chain | 25 |
| | Amino acid sequence encoding the variable region of the heavy chain | 26 |
| | Nucleotide sequence encoding the variable region of the light chain | 27 |
| | Amino acid sequence encoding the variable region of the light chain | 28 |
| 140 | Nucleotide sequence encoding the variable region of the heavy chain | 29 |
| | Amino acid sequence encoding the variable region of the heavy chain | 30 |
| | Nucleotide sequence encoding the variable region of the light chain | 31 |
| | Amino acid sequence encoding the variable region of the light chain | 32 |
| 171 | Nucleotide sequence encoding the variable region of the heavy chain | 33 |
| | Amino acid sequence encoding the variable region of the heavy chain | 34 |
| | Nucleotide sequence encoding the variable region of the light chain | 35 |
| | Amino acid sequence encoding the variable region of the light chain | 36 |
| 179 | Nucleotide sequence encoding the variable region of the heavy chain | 37 |
| | Amino acid sequence encoding the variable region of the heavy chain | 38 |
| | Nucleotide sequence encoding the variable region of the light chain | 39 |
| | Amino acid sequence encoding the variable region of the light chain | 40 |
| 188 | Nucleotide sequence encoding the variable region of the heavy chain | 41 |
| | Amino acid sequence encoding the variable region of the heavy chain | 42 |
| | Nucleotide sequence encoding the variable region of the light chain | 43 |
| | Amino acid sequence encoding the variable region of the light chain | 44 |
| 199 | Nucleotide sequence encoding the variable region of the heavy chain | 45 |
| | Amino acid sequence encoding the variable region of the heavy chain | 46 |
| | Nucleotide sequence encoding the variable region of the light chain | 47 |
| | Amino acid sequence encoding the variable region of the light chain | 48 |
| 213 | Nucleotide sequence encoding the variable region of the heavy chain | 49 |
| | Amino acid sequence encoding the variable region of the heavy chain | 50 |
| | Nucleotide sequence encoding the variable region of the light chain | 51 |
| | Amino acid sequence encoding the variable region of the light chain | 52 |

Antibody Therapeutics

Anti-Ten-M2 antibodies can have therapeutic effects in treating symptoms and conditions related to Ten-M2 activity. For example, the antibodies can inhibit the formation of the Ten-M2/Ten-M2 duplex, thereby inhibiting cancer metastasis, or the antibodies can be associated with an agent and deliver a lethal toxin to a targeted cell. In addition, the anti-Ten-M2 antibodies are useful as diagnostics for the disease states, especially cancer and the metastasis of cancer.

If desired, the isotype of an anti-Ten-M2 antibody can be switched, for example to take advantage of a biological property of a different isotype. For example, in some circumstances it may be desirable in connection with the generation of antibodies as therapeutic antibodies against Ten-M2 that the antibodies be capable of fixing complement and participating in complement-dependent cytotoxicity (CDC). There are a number of isotypes of antibodies that are capable of the same, including, without limitation, the following: murine IgM, murine IgG2a, murine IgG2b, murine IgG3, human IgM, human IgG1, and human IgG3. It will be appreciated that antibodies that are generated need not initially possess such an isotype but, rather, the antibody as generated can possess any isotype and the antibody can be isotype switched thereafter using conventional techniques that are well known in the art. Such techniques include the use of direct recombinant techniques (see e.g., U.S. Pat. No. 4,816,397), cell-cell fusion techniques (see e.g., U.S. Pat. Nos. 5,916,771 and 6,207,418), among others.

By way of example, the anti-Ten-M2 antibodies discussed herein are human antibodies. If an antibody possessed desired binding to Ten-M2, it could be readily isotype switched to generate a human IgM, human IgG1, or human IgG3 isotype, while still possessing the same variable region (which defines the antibody's specificity and some of its affinity). Such molecule would then be capable of fixing complement and participating in CDC.

In the cell-cell fusion technique, a myeloma or other cell line is prepared that possesses a heavy chain with any desired isotype and another myeloma or other cell line is prepared that possesses the light chain. Such cells can, thereafter, be fused and a cell line expressing an intact antibody can be isolated.

Accordingly, as antibody candidates are generated that meet desired “structural” attributes as discussed above, they can generally be provided with at least certain of the desired “functional” attributes through isotype switching.

Biologically active antibodies that bind Ten-M2 are preferably used in a sterile pharmaceutical preparation or formulation to reduce the activity of Ten-M2. Anti-Ten-M2 antibodies preferably possess adequate affinity to potently suppress Ten-M2 activity to within the target therapeutic range. The suppression can result from the ability of the antibody to interfere with the binding of Ten-M2 to another Ten-M2 protein. Additionally, the antibodies can alter the conformation or the Ten-M2 proteins so that Ten-M2 signaling events do not generally occur.

When used for in vivo administration, the antibody formulation is preferably sterile. This is readily accomplished by any method know in the art, for example by filtration through sterile filtration membranes. The antibody ordinarily will be stored in lyophilized form or in solution. Sterile filtration may be performed prior to or following lyophilization and reconstitution.

Therapeutic antibody compositions generally are placed into a container having a sterile access port, for example, an intravenous solution bag or vial having an adapter that allows retrieval of the formulation, such as a stopper pierceable by a hypodermic injection needle.

The route of antibody administration is in accord with known methods, e.g., injection or infusion by intravenous, intraperitoneal, intracerebral, intramuscular, intraocular, intraarterial, intrathecal, inhalation or intralesional routes, or by sustained release systems as noted below. In some situations the antibody is preferably administered by infusion or by bolus injection. In other situations a therapeutic composition comprising the antibody can be administered through the nose or lung, preferably as a liquid or powder aerosol (lyophilized). The composition may also be administered intravenously, parenterally or subcutaneously as desired. When administered systemically, the therapeutic composition should be sterile, pyrogen-free and in a parenterally acceptable solution having due regard for pH, isotonicity, and stability. These conditions are known to those skilled in the art.

Antibodies for therapeutic use, as described herein, are typically prepared with suitable carriers, excipients, and other agents that are incorporated into formulations to provide improved transfer, delivery, tolerance, and the like. Briefly, dosage formulations of the antibodies described herein are prepared for storage or administration by mixing the antibody having the desired degree of purity with one or more physiologically acceptable carriers, excipients, or stabilizers. These formulations may include, for example, powders, pastes, ointments, jellies, waxes, oils, lipids, lipid (cationic or anionic) containing vesicles (such as Lipofectin™), DNA conjugates, anhydrous absorption pastes, oil-in-water and water-in-oil emulsions, carbowax (polyethylene glycols of various molecular weights), semi-solid gels, and semi-solid mixtures containing carbowax. The formulation may include buffers such as TRIS HCl, phosphate, citrate, acetate and other organic acid salts; antioxidants such as ascorbic acid; low molecular weight (less than about ten residues) peptides such as polyarginine, proteins, such as serum albumin, gelatin, or immunoglobulins; hydrophilic polymers such as polyvinylpyrrolidinone; amino acids such as glycine, glutamic acid, aspartic acid, or arginine; monosaccharides, disaccharides, and other carbohydrates including cellulose or its derivatives, glucose, mannose, or dextrins; chelating agents such as EDTA; sugar alcohols such as mannitol or sorbitol; counterions such as sodium and/or nonionic surfactants such as TWEEN, PLURONICS or polyethyleneglycol. Other acceptable carriers, excipients and stabilizers are well known to those of skill in the art. Any of the foregoing mixtures may be appropriate in treatments and therapies in accordance with the present invention, provided that the active ingredient in the formulation is not inactivated by the formulation and the formulation is physiologically compatible and tolerable with the route of administration. See also Baldrick P. “Pharmaceutical excipient development: the need for preclinical guidance.” Regul. Toxicol. Pharmacol. 32(2):210-8 (2000), Wang W. “Lyophilization and development of solid protein pharmaceuticals.” Int. J. Pharm. 203(1-2):1-60 (2000), Charman WN “Lipids, lipophilic drugs, and oral drug delivery-some emerging concepts.” J Pharm Sci .89(8):967-78 (2000), Powell et al. “Compendium of excipients for parenteral formulations” PDA J Pharm Sci Technol. 52:238-311 (1998) and the citations therein for additional information.

Sterile compositions for injection can be formulated according to conventional pharmaceutical practice as described in Remington: The Science and Practice of Pharmacy (20^{th} ed, Lippincott Williams & Wilkens Publishers (2003)). For example, dissolution or suspension of the active compound in a vehicle such as water or naturally occurring vegetable oil like sesame, peanut, or cottonseed oil or a synthetic fatty vehicle like ethyl oleate or the like may be desired. Buffers, preservatives, antioxidants and the like can be incorporated according to accepted pharmaceutical practice.

The antibodies can also be administered in and released over time from sustained-release preparations. Suitable examples of sustained-release preparations include semipermeable matrices of solid hydrophobic polymers containing the polypeptide. The matrices may be in the form of shaped articles, films or microcapsules. Examples of sustained-release matrices include polyesters, hydrogels (e.g., poly(2-hydroxyethyl-methacrylate) as described by Langer et al., J. Biomed Mater. Res., (1981) 15:167-277 and Langer, Chem. Tech., (1982) 12:98-105, or poly(vinylalcohol)), polylactides (U.S. Pat. No. 3,773,919, EP 58,481), copolymers of L-glutamic acid and gamma ethyl-L-glutamate (Sidman et al., Biopolymers, (1983) 22:547-556), non-degradable ethylene-vinyl acetate (Langer et al., supra), degradable lactic acid-glycolic acid copolymers such as the LUPRON Depot™ (injectable microspheres composed of lactic acid-glycolic acid copolymer and leuprolide acetate), and poly-D-(−)-3-hydroxybutyric acid (EP 133,988).

While polymers such as ethylene-vinyl acetate and lactic acid-glycolic acid enable release of molecules for over 100 days, certain hydrogels release proteins for shorter time periods. When encapsulated proteins remain in the body for a long time, they may denature or aggregate as a result of exposure to moisture at 37° C., resulting in a loss of biological activity and possible changes in immunogenicity. Rational strategies can be devised for protein stabilization depending on the mechanism involved. For example, if the aggregation mechanism is discovered to be intermolecular S—S bond formation through disulfide interchange, stabilization may be achieved by modifying sulfhydryl residues, lyophilizing from acidic solutions, controlling moisture content, using appropriate additives, and developing specific polymer matrix compositions.

Sustained-released compositions also include preparations of crystals of the antibody suspended in suitable formulations capable of maintaining crystals in suspension. These preparations when injected subcutaneously or intraperitonealy can produce a sustained release effect. Other compositions also include liposomally entrapped antibodies. Liposomes containing such antibodies are prepared by methods known per se: U.S. Pat. No. DE 3,218,121; Epstein et al., Proc. Natl. Acad. Sci. USA, (1985) 82:3688-3692; Hwang et al., Proc. Natl. Acad. Sci. USA, (1980) 77:4030-4034; EP 52,322; EP 36,676; EP 88,046; EP 143,949; 142,641; Japanese patent application 83-118008; U.S. Pat. Nos. 4,485,045 and 4,544,545; and EP 102,324.

The dosage of the antibody formulation for a given patient may be determined by the attending physician. In determining the appropriate dosage the physician may take into consideration various factors known to modify the action of therapeutics, including, for example, severity and type of disease, body weight, sex, diet, time and route of administration, other medications and other relevant clinical factors. Therapeutically effective dosages may be determined by either in vitro or in vivo methods.

An effective amount of the antibodies, described herein, to be employed therapeutically will depend, for example, upon the therapeutic objectives, the route of administration, and the condition of the patient. Accordingly, it is preferred for the therapist to titer the dosage and modify the route of administration as required to obtain the optimal therapeutic effect. A typical daily dosage might range from about 0.001 mg/kg to up to 100 mg/kg or more, depending on the factors mentioned above. Typically, the clinician will administer the therapeutic antibody until a dosage is reached that achieves the desired effect. The progress of this therapy is easily monitored by conventional assays.

It is expected that the antibodies described herein will have therapeutic effect in treatment of symptoms and conditions resulting from or related to the activity of Ten-M2.

Design and Generation of Other Therapeutics

In accordance with the present invention and based on the activity of the antibodies that are produced and characterized herein with respect to Ten-M2, the design of other therapeutic modalities is facilitated and disclosed to one of skill in the art. Such modalities include, without limitation, advanced antibody therapeutics, such as bispecific antibodies, immunotoxins, and radiolabeled therapeutics, generation of peptide therapeutics, gene therapies, particularly intrabodies, antisense therapeutics, and small molecules.

In connection with the generation of advanced antibody therapeutics, where complement fixation is a desirable attribute, it cam be possible to sidestep the dependence on complement for cell killing through the use of bispecifics, immunotoxins, or radiolabels, for example.

For example, bispecific antibodies can be generated that comprise (i) two antibodies, one with a specificity to Ten-M2 and another to a second molecule, that are conjugated together, (ii) a single antibody that has one chain specific to Ten-M2 and a second chain specific to a second molecule, or (iii) a single chain antibody that has specificity to both Ten-M2 and the other molecule. Such bispecific antibodies can be generated using techniques that are well known; for example, in connection with (i) and (ii) see e.g., Fanger et al. Immunol Methods 4:72-81 (1994) and Wright and Harris, supra. and in connection with (iii) see e.g., Traunecker et al. Int. J Cancer (Suppl.) 7:51-52 (1992). In each case, the second specificity can be made as desired. For example, the second specificity can be made to the heavy chain activation receptors, including, without limitation, CD16 or CD64 (see e.g., Deo et al. 18:127 (1997)) or CD89 (see e.g., Valerius et al. Blood 90:4485-4492 (1997)). In some embodiments, the antibodies are designed so as to bind to two Ten-M2 proteins. In some embodiments, the antibodies are designed so as to bind to two Ten-M2 proteins, and to further prevent the two Ten-M2 proteins from actually contacting one another in a manner so as to allow signaling to occur. In this embodiment, the result can be beneficial in that the Ten-M2 is being prevented from signaling by the antibody, and each antibody can stop two Ten-M2 molecules.

Antibodies can also be modified to act as immunotoxins utilizing techniques that are well known in the art. See e.g., Vitetta Immunol Today 14:252 (1993). See also U.S. Pat. No. 5,194,594. In connection with the preparation of radiolabeled antibodies, such modified antibodies can also be readily prepared utilizing techniques that are well known in the art. See e.g., Junghans et al. in Cancer Chemotherapy and Biotherapy 655-686 (2d edition, Chafner and Longo, eds., Lippincott Raven (1996)). See also U.S. Pat. Nos. 4,681,581, 4,735,210, 5,101,827, 5,102,990 (RE 35,500), 5,648,471, and 5,697,902. Each of immunotoxins and radiolabeled molecules would be likely to kill cells expressing the desired multimeric enzyme subunit oligomerization domain. In some embodiments, a pharmaceutical composition comprising an effective amount of the antibody in association with a pharmaceutically acceptable carrier or diluent is provided.

In some embodiments, an anti-Ten-M2 antibody is linked to an agent (e.g., radioisotope, pharmaceutical composition, or a toxin). Preferably, such antibodies can be used for the treatment of diseases, such diseases can relate to the over or under expression of ten-M proteins and Ten-M2 in particular. For example, it is contemplated that the drug possesses the pharmaceutical property selected from the group of antimitotic, alkylating, antimetabolite, antiangiogenic, apoptotic, alkaloid, COX-2, and antibiotic agents and combinations thereof. The drug can be selected from the group of nitrogen mustards, ethylenimine derivatives, alkyl sulfonates, nitrosoureas, triazenes, folic acid analogs, anthracyclines, taxanes, COX-2 inhibitors, pyrimidine analogs, purine analogs, antimetabolites, antibiotics, enzymes, epipodophyllotoxins, platinum coordination complexes, vinca alkaloids, substituted ureas, methyl hydrazine derivatives, adrenocortical suppressants, antagonists, endostatin, taxols, camptothecins, oxaliplatin, doxorubicins and their analogs, and a combination thereof.

Examples of toxins further include gelonin, Pseudomonas exotoxin (PE), PE40, PE38, diphtheria toxin, ricin, ricin, abrin, alpha toxin, saporin, ribonuclease (RNase), DNase I, Staphylococcal enterotoxin-A, pokeweed antiviral protein, gelonin, Pseudomonas endotoxin, as well as derivatives, combinations and modifications thereof.

Examples of radioisotopes include gamma-emitters, positron-emitters, and x-ray emitters that may be used for localization and/or therapy, and beta-emitters and alpha-emitters that may be used for therapy. The radioisotopes described previously as useful for diagnostics, prognostics and staging are also useful for therapeutics. Non-limiting examples of anti-cancer or anti-leukemia agents include anthracyclines such as doxorubicin (adriamycin), daunorubicin (daunomycin), idarubicin, detorubicin, carminomycin, epirubicin, esorubicin, and morpholino and substituted derivatives, combinations and modifications thereof. Exemplary pharmaceutical agents include cis-platinum, taxol, calicheamicin, vincristine, cytarabine (Ara-C), cyclophosphamide, prednisone, daunorubicin, idarubicin, fludarabine, chlorambucil, interferon alpha, hydroxyurea, temozolomide, thalidomide, and bleomycin, and derivatives, combinations and modifications thereof. Preferably, the anti-cancer or anti-leukemia is doxorubicin, morpholinodoxorubicin, or morpholinodaunorubicin.

As will be appreciated by one of skill in the art, in the above embodiments, while affinity values can be important, other factors can be as important or more so, depending upon the particular function of the antibody. For example, for an immunotoxin (toxin associated with an antibody), the act of binding of the antibody to the target can be useful; however, in some embodiments, it is the internalization of the toxin into the cell that is the desired end result. As such, antibodies with a high percent internalization can be desirable in these situations. However, they need not be desirable if the antibody is to prevent duplex formation of the Ten-M2 protein with another Ten-M2 protein. Thus, in one embodiment, antibodies with a high efficiency in internalization are contemplated. A high efficiency of internalization can be measured as a percent internalized antibody, and can be from a low value to 100%. For example, in varying embodiments, 0.1-5, 5-10, 10-20, 20-30, 30-40, 40-45, 45-50, 50-60, 60-70, 70-80, 80-90, 90-99, and 99-100% can be a high efficiency. As will be appreciated by one of skill in the art, the desirable efficiency can be different in different embodiments, depending upon, for example, the associated agent, the amount of antibody that can be administered to an area, the side effects of the antibody-agent complex, the type (e.g., cancer type) and severity of the problem to be treated.

In other embodiments, the antibodies disclosed herein provide an assay kit for the detection of Ten-M2 protein in mammalian tissues or cells in order to screen for a disease or disorder associated with changes in levels of Ten-M2. The kit comprises an antibody that binds the antigen protein and means for indicating the reaction of the antibody with the antigen, if present.

In some embodiments, an article of manufacture is provided comprising a container, comprising a composition containing an anti-Ten-M2 antibody, and a package insert or label indicating that the composition can be used to treat disease mediated by Ten-M2. Preferably a mammal and, more preferably, a human, receives the anti-Ten-M2 antibody.

### EXAMPLES

The following examples, including the experiments conducted and results achieved are provided for illustrative purposes only and are not to be construed as limiting upon the invention described herein.

### Example 1

### Generation of Anti-Ten-M2 Antibodies

Monoclonal antibodies against Ten-M2 were developed by immunizing XenoMouse® mice (IgG2 Kappa XenoMouse Strain), Abgenix, Inc. Fremont, Calif.) using antigens with the sequences depicted in FIGS. 1A and 1B. The antigen depicted in FIG. 1A also had a V5-6xHis (shown double underlined and italicized) and human Fc tags (shown bolded) added, and the antigen depicted in FIG. 1B also had a 6xHis-V5 tag (shown italicized and double underlined) added. The signal peptide is underlined.

Hybridomas and B cell clones produced from the above immunized mice were screened for Ten-M2 specific monoclonal antibodies. The ELISA plates were coated with soluble Ten-M2 antigen (see FIG. 1B) and incubated at 4° C. overnight. After the incubation, the plates were washed with Washing Buffer (0.05% Tween 20 in PBS) 3 times. Blocking Buffer (200 μL/well, 0.5% BSA, 0.1% Tween 20, 0.01% Thimerosal in lx PBS) was added and the plates were incubated at room temperature for 1 hour. After incubation, the plates were washed with Washing Buffer three times. Hybridoma or B cell clone supernatant (50 μL/well), positive and negative controls were added and the plates were incubated at room temperature for 2 hours

After incubation, the plates were washed three times with Washing Buffer. Goat anti-huIgGfc-HRP detection antibody (100 μL/well) was added and the plates were incubated at room temperature for 1 hour. After the incubation, the plates were washed three times with Washing Buffer. TMB substrate (100 μL/well) was added and the plates allowed to develop for about 10 minutes (until negative control wells barely started to show color), then 50 μL/well of stop solution was added and the plates read on an ELISA plate reader at wavelength 450nm.

Hybridoma or B cell clone supernatants that were identified as positive for binding in the above ELISA were then assayed for the ability to bind to endogenously expressed Ten-M2 using the SNB-19 cell line which naturally expresses the antigen. A FMAT based fluorescence assay was performed for 247 B cell clone samples identified in the screen above. Briefly, SNB-19 cells were seeded at 10,000 cells per well in a 96-well microtiter dish. After the cells had adhered, the media was removed and replaced with B cell clone supernatant. After a one hour incubation, the cells were washed and the bound antibody was detected via a Cy5-conjugated anti-Human IgGFc specific polyclonal antibody. Positive wells were imaged using the FMAT reader. Table 2, below, summarizes the number of Hybridomas and B cell clones that bound to the soluble Ten-M2 (Cur007) ECD and the number of B cell clones which then also bound to the SNB-19 cell line.

TABLE 2
Anti-Ten-M2 antibodies binding to soluble and cell surface Ten-M2

| | |
|---|---|
| Number of Curagen 007-V5HIS reactive Hybridoma clones | 6 |
| Number of Curagen 007-V5HIS Reactive B cell clones | 247 |
| Number of B cell clones which bound to SNB-19 cell line (via FMAT and FACS) | 93 |

All of the B cell clones were tested for their binding to a V5-His soluble peptide, and none of the 247 cross-reacted to it. The 6 hybridoma clones were only tested for binding to soluble Ten-M2-V5-His protein, and did not progress beyond this assay. Their sequences are provided in FIGS. 5 to 9.

### Example 2

### Binding Specificity of Ten-M2 Antibodies:

This example demonstrates the specificity of the various antibodies generated. The antibodies were tested for their ability to bind to Ten-M3 (Cur026) expressing stable cell line and Ten-M4 (CR105) expressed endogenously on a cancer cell line. A FMAT based fluorescence assay was performed for the 247 B cell clone samples identified in the screen above. Briefly, cells were seeded at 10,000 cells per well in a 96-well microtiter dish. After the cells had adhered, the media was removed and replaced with B cell clone supernatant. After a one hour incubation, the cells were washed and the bound antibody detected via a Cy5-conjugated anti-Human IgGFc specific polyclonal antibody. Positive wells were imaged using the FMAT reader. Table 3 below, summarizes the data demonstrating that only one antibody, 179, cross-reacts to Ten-M3.

TABLE 3
Binding Profile of anti-Ten-M2 antibodies to related homologues
Ten-M3 (CUR026) and Ten-M4 (CUR105).

| | Cur026 stable cell line | | | PC3 cell line (Cur105) | | |
|---|---|---|---|---|---|---|
| Antibody ID | X Mean | X Geo Mean | Crossreact? | X Mean | X Geo Mean | Crossreact? |
| 120 | 3.99 | 3.42 | NO | 3.96 | 3.34 | NO |
| 140 | 3.66 | 3.13 | NO | 4.04 | 3.45 | NO |
| 171 | 5.12 | 3.64 | NO | 3.84 | 3.26 | NO |
| 179 | 22.75 | 8.05 | YES | 3.87 | 3.28 | NO |
| 188 | 3.7 | 3.16 | NO | 4.47 | 3.5 | NO |
| 199 | 3.8 | 3.25 | NO | 3.86 | 3.27 | NO |
| 213 | 3.45 | 3.01 | NO | 3.92 | 3.33 | NO |

As can be observed from the data above, the described antibodies demonstrated an increase in binding relative to the background level. These results demonstrated that the antibodies can be relatively selective or specific for binding to Ten-M2 over other, closely related antigens and proteins.

### Example 3

### Internalization Assays of Various Ten-M2 Antibodies

This example demonstrated that various Ten-M2 antibodies could be internalized within SNB-19 cells. As reported below, several of the antibodies were internalized at fairly high levels of efficiency (e.g., a relatively large amount of the antibody can be internalized by the cells).

The Ten-M2 antibodies were used to stain SNB-19 cells. This was done first at 4° Celsius (resulting in no internalization for a background measurement), and was then shifted to 37° Celsius for 30 minutes to induce internalization.

SNB-19 cells were removed from culture dishes using Cell Dissociation Media (Sigma), counted, and transferred (100,000 cells) to a 96-well VEE bottom plate. The cells were spun down, the media removed, and the cells resuspended with 100 μL of hybridoma supernatant and incubated for 30 minutes on ice. The incubated cells were spun down, and bound antibody was detected using a secondary antibody which had been linked to Alex647 dye via a disulphide linkage (anti-Hu IgG Fc-SS-Alexa 647 or anti-Hu IgG Fab-SS-Alexa647 @ 1 μg/ml). The secondary antibody was incubated for 7 minutes on ice. After the incubation, cells were washed and resuspended with ice cold 10% FCS/PBS. The sample was then split into three samples, which were spun down, and the supernatant removed.

Two of the replicates were resuspended with ice cold 10% FCS/PBS and then incubated on ice for 30 minutes. The other replicate was resuspended with warm 10% FCS/PBS and incubated @ 37° C. for 30 minutes. After the 30 minute incubation, cells were spun down and resuspended with one of the following buffers. One buffer was 250 μL of cold 50mM Glutathione, which was added to the 4° C. sample. This was used as a measure of background fluorescence due to incomplete reduction of the disulphide bond. The second buffer had 250 μL of cold 50mM Glutathione, which was added to a 37° Celsius replicate. The Glutathione only had access to cell surface secondary antibodies. If the antibody was internalized the disulfide bond would not have been reduced by the Glutathione and the cell would have remained fluorescent. The remaining fluorescent intensity was therefore proportional to the amount of internalization of the antibody. The third buffer was 250 μL of cold 10% FCS/PBS, which was added to the other 4° C. sample. This sample was a control to show the maximum fluorescence.

The samples were then incubated on ice for 30 minutes, spun down, and resuspended with 300 μL of ice cold 10% FCS/PBS and analyzed by Flow Cytometry. The results are displayed in Table 4.

TABLE 4
Internalization: Ten-M2 (Cur007) specific antibodies used to stain SNB-19 cell line at 4° C.
(no internalization) and then shifted to 37° C. for 30 minutes to induce internalization

| Sample | Total Bound Fluorescence | Internalized Fluorescence | Background Fluorescence | % Internalization | Above Background |
|---|---|---|---|---|---|
| Cell alone | 3.25 | 2.43 | 2.13 | n/a | |
| 2° Ab alone | 2.65 | 2.3 | 2.06 | n/a | |
| isotype control Ab | 2.2 | 2.2 | 2.02 | n/a | |
| 120 | 58.39 | 26.38 | 5.27 | 40% | YES |
| 140 | 75.32 | 36.49 | 6.36 | 44% | YES |
| 171 | 85.78 | 38.3 | 7.68 | 39% | YES |
| 179 | 89.62 | 13.63 | 5.07 | 29% | YES |
| 188 | 41.65 | 22.13 | 4.42 | 48% | YES |
| 199 | 64.75 | 32.87 | 5.7 | 46% | YES |
| 213 | 79.68 | 40.17 | 6.68 | 46% | YES |

As can be observed in the above table, all of the antibodies tested were internalized some extent. The minimal percent internalization was 29%. Several antibodies were internalized at over 40%, and one antibody, 188, was internalized at about 50% internalization.

### Example 4

### Antibody Toxin Conjugates

This example demonstrates how an antibody conjugated to a toxin was used as an effective composition to prevent cancer cells from proliferating. A clonogenic assay was used to determine whether primary antibodies could induce cancer cell death when the antibody was conjugated with a saporin toxin conjugated secondary antibody reagent. (For example, as described in Kohls and Lappi, “Mab-ZAP: A tool for evaluating antibody efficacy for use in an immunotoxin,” Biotechniques, 28(1):162-5 (Jan. 2000), hereby incorporated by reference in its entirety).

Briefly, cells were plated onto flat bottom tissue culture plates at a density of about 3000 cells per well. On day 2 or when cells reached ˜25% confluency, 100 ng/well secondary mAb-toxin (goat anti-human IgG-saporin; Advanced Targeting Systems; HUM-ZAP; cat. no. IT-22) was added. An anti-EGFR antibody (positive control), anti-Ten-M2 mAb, or an isotype control mAb was then added to each well at the desired concentration (typically 1 to 500 ng/mL). On day 5, the cells were trypsinized, transferred to a 6-well tissue culture dish, and incubated at 37° C. Plates were examined daily. On days 10-12, the plates were Giemsa stained and colonies on the plates were counted. Plating efficiency was determined by the number of colonies that eventually formed.

The cytotoxic chemotherapy reagent 5 Flurouracil (5-FU) was used as the positive control and induced almost complete killing, whereas the saporin conjugated-goat anti-human secondary antibody alone had little effect. A monoclonal antibody (NeoMarkers MS-269-PABX) generated against the EGF-like receptor expressed by both cell lines was used to demonstrate primary antibody- and secondary antibody-saporin conjugate specific killing.

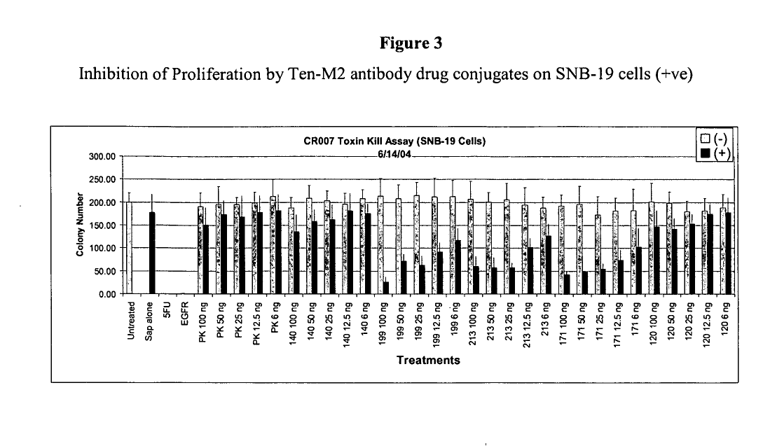

Various concentrations, (e.g., between 5 and 1000 pM) of the antibody/toxin conjugates were administered to SNB-19 cells under conditions to allow for the internalization of the antibody/toxin conjugates. The cells were then allowed to continue growing for 96 hours. The colonies were then counted to determine the amount of inhibition of SNB- 19 cell growth. The results are presented in FIG. 3.

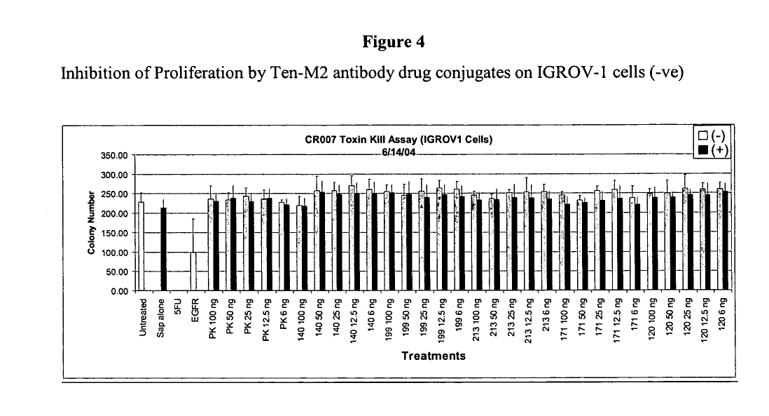

As can be observed in the FIG., several anti-Ten-M2 antibodies resulted in 50% or more reduction in the concentration of SNB-19 cells at amounts ranging between 6 and 100 ng. FIG. 4 shows an inhibition of proliferation assay using a cancer cell line, IGROV-1, that does not express Ten-M2. As expected, growth of IGROV-1 cells was not affected by the addition of the anti-Ten-M2 antibodies, indicating that the growth inhibition of SNB-19 cells seen in FIG. 3 was due to the specific nature of the anti-Ten-M2 antibodies.

### Example 5

### Structural Analysis of Anti-Ten-M2 Antibodies

The variable heavy chains and the variable light chains for the anti-Ten-M2 antibodies were sequenced to determine their DNA sequences. The complete sequence information for all anti-Ten-M2 antibodies are shown in FIGS. 5 through 17 with nucleotide and amino acid sequences for each gamma and kappa chain combination.

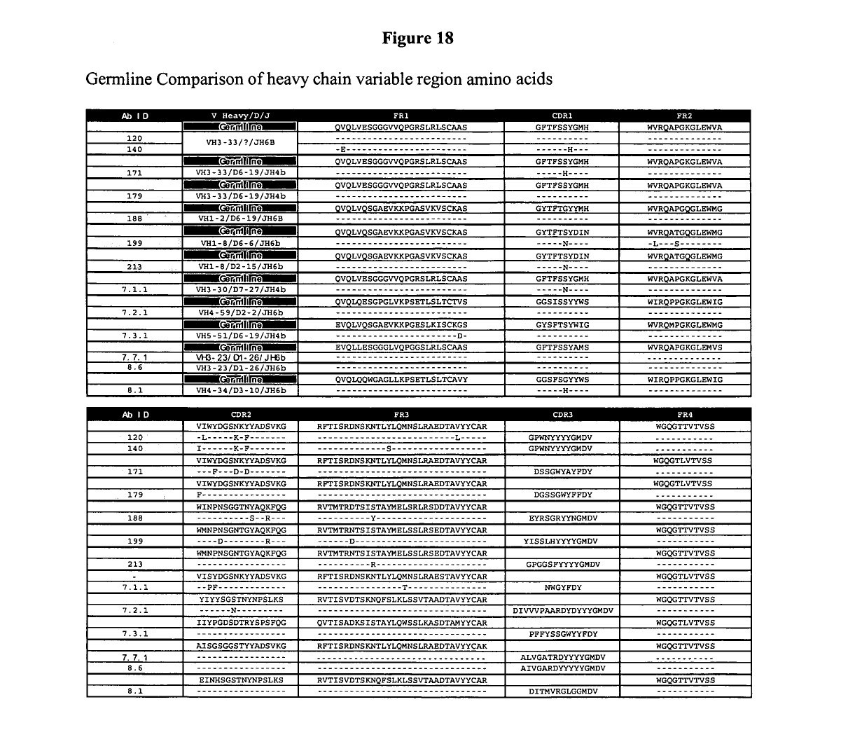

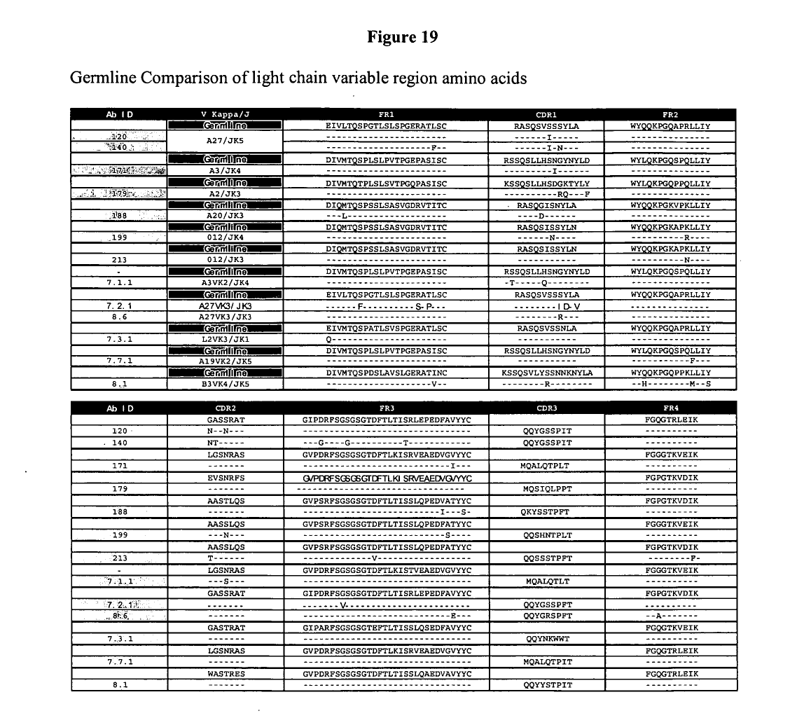

The variable heavy chain nucleotide sequences were analyzed to determine the VH family, the D-region sequence and the J-region sequence. The sequences were then translated to determine the primary amino acid sequence and compared to the germline VH, D and J-region sequences to assess somatic hypermutations. The primary amino acid sequences of all the anti-Ten-M2 heavy chains are shown in FIG. 18. The germline sequences are shown above and the mutations are indicated with the new amino acid sequence. Amino acids in the sequence that are identical to the indicated germline sequence are indicated with a dash (-). The light chain was analyzed similarly to determine the V and the J-regions and to identify any somatic mutations from germline light chain sequences (FIG. 19).

### Example 6

### V Gene Usage of Various Antibodies

This example demonstrates the various V genes that are associated with the particular antibodies characterized above. The V genes in many of the antibodies were analyzed to determine which genes had been employed in the particular antibodies. The V genes involved in both the heavy and the light chains of the antibodies are presented in Table 5 below.

TABLE 5

| | Heavy Chain | | | Light Chain | |
|---|---|---|---|---|---|
| Chain Name | V | D | J | V | J |
| 120 | VH3-33 | | JH6B | A27 | JK5 |
| 140 | VH3-33 | | JH6B | A27 | JK5 |
| 171 | VH3-33 | D6-19 | JH4B | A3 | JK4 |
| 179 | VH3-33 | D6-19 | JH4B | A2 | JK3 |
| 188 | VH1-2 | D6-19 | JH6B | A20 | JK3 |
| 199 | VH1-8 | D6-6 | JH6B | O12 | JK4 |
| 213 | VH1-8 | D2-15 | JH6B | O12 | JK3 |
| 7.1.1 | VH3-30 | D7-27 | JH4B | A3VK2 | JK4 |
| 7.2.1 | VH4-59 | D2-2 | JH6B | A27VK3 | JK3 |
| 7.3.1 | VH5-51 | D6-19 | JH4B | A27VK3 | JK3 |
| 7.7.1 | VH3-23 | D1-26 | JH6B | L2VK3 | JK1 |
| 8.6 | VH3-23 | D1-26 | JH6B | A19VK2 | JK5 |
| 8.1 | VH4-34 | D3-10 | JH6B | B3VK4 | JK5 |

As can be seen in the table above, all of the antibodies involved the JH6B or JH4B genes.

### Example 7

### Epitope Binning and BiaCore® Affinity Determination

Epitope Binning

Certain antibodies, described herein are “binned” in accordance with the protocol described in U.S. Patent Application Publication No. 20030157730. MxhIgG conjugated beads are prepared for coupling to primary antibody. The volume of supernatant needed is calculated using the following formula: (n+10)×50 μL (where n =total number of samples on plate). Where the concentration is known, 0.5 μg/mL is used. Bead stock is gently vortexed, then diluted in supernatant to a concentration of 2500 of each bead per well or 0.5×10⁵/mL and incubated on a shaker in the dark at RT overnight, or 2 hours if at a known concentration of 0.5 μg/mL. Following aspiration, 50 μL of each bead is added to each well of filter plate, then washed once by adding 100 μL/well wash buffer and aspirating. Antigen and controls are added to filter plate 50 μL/well then covered and allowed to incubate in the dark for 1 hour on shaker. Following a wash step, a secondary unknown antibody is added at 50 μL/well using the same dilution (or concentration if known) as is used for the primary antibody. The plates are then incubated in the dark for 2 hours at RT on shaker followed by a wash step. Next, 50 μL/well biotinylated mxhIgG diluted 1:500 is added and allowed to incubate in the dark for 1hour on shaker at RT. Following a wash step, 50 μL/well Streptavidin-PE is added at 1:1000 and allowed to incubate in the dark for 15 minutes on shaker at RT. Following a wash step, each well is resuspended in 80 μL blocking buffer and read using Luminex. Results show that the monoclonal antibodies belong to distinct bins. Competitive binding by antibodies from different bins supports antibody specificity for similar or adjacent epitopes. Non competitive binding supports antibody specificity for unique epitopes.

Determination of Anti-Ten-M2 mAb Affinity Using BiaCore® Analysis

BiaCore® analysis was used to determine binding affinity of anti-Ten-M2 antibody to Ten-M2 antigen. The analysis was performed at 25° C. using a BiaCore® biosensor equipped with a research-grade CM5 sensor chip. A high-density goat a human antibody surface over a CM5 BiaCore® chip was prepared using routine amine coupling. Antibody supernatants were diluted to ˜5 μg/mL in HBS-P running buffer containing 100 μg/mL BSA and 10 mg/mL carboxymethyldextran. The antibodies were then captured individually on a separate surface using a 2 minute contact time, and a 5 minute wash for stabilization of antibody baseline.

Ten-M2 antigen was injected at 292 nM over each surface for 75 seconds, followed by a 3-minute dissociation. Double-referenced binding data were obtained by subtracting the signal from a control flow cell and subtracting the baseline drift of a buffer inject just prior to the Ten-M2 injection. Ten-M2 binding data for each mAb were normalized for the amount of mAb captured on each surface. The normalized, drift-corrected responses were also measured. The kinetic analysis results of anti-Ten-M2 mAB binding at 25° C. are listed in Table 7 below.

TABLE 7
Ten-M2 Low Resolution BiaCore ® Screen of 6 Purified
TEN-M2 mABs

| | R_{L} of antibody | | | |
|---|---|---|---|---|
| mAB | immobilized | Kₐ (M⁻¹s⁻¹) | K_{d}(s⁻¹) | K_{D} (nM) |
| 120 | 1096 | 2.62 × 10⁴ | 4.65 × 10⁻⁴ | 18* |
| 140 | 1040 | 4.01 × 10⁴ | 6.05 × 10⁻⁴ | 15* |
| 171 | 1075 | 3.26 × 10⁴ | 1.00 × 10^{-6$} | 0.031 |
| 179 | 928 | 1.63 × 10⁴ | 1.00 × 10^{-5$} | 0.61 |
| 199 | 1031 | 2.33 × 10⁴ | 1.00 × 10^{-6$} | 0.043 |
| 213 | 1119 | 2.90 × 10⁴ | 3.22 × 10⁻⁵ | 1.1 |

^{$}k_{d} held constant at this value in the non-linear fitting process.
*Extremely complex kinetics

### Example 8

### Detection of Ten-M2 Protein by Anti-Ten-M2 mAB by Western Blot Analysis Immunohistochemistry and FACS Analysis

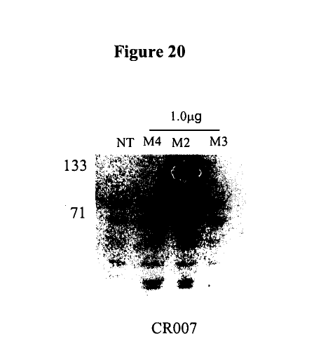

To determine the antigen binding properties and cross reactivities of anti-Ten-M2 monoclonal antibodies, one μg of Ten-M2 (M2), Ten-M3 (M3), or Ten-M4 (M4)recombinant protein (R&D systems) were loaded under reducing conditions on 4-20% Tris-glycine gels (Invitrogen) and electrophoretically transferred to 0.45 μpm PVDF membranes (Invitrogen). Membranes were blocked with 3% BSA (Sigma, St. Louis, MO) in TBST for 3 hrs and probed with TEN-M2 antibody at a concentration of 2 μg/ml for 3 hrs. As shown in FIG. 20, TEN-M2 mAb specifically recognizes the p125 Ten-M2 species, but not Ten-M3 or Ten-M4 protein.

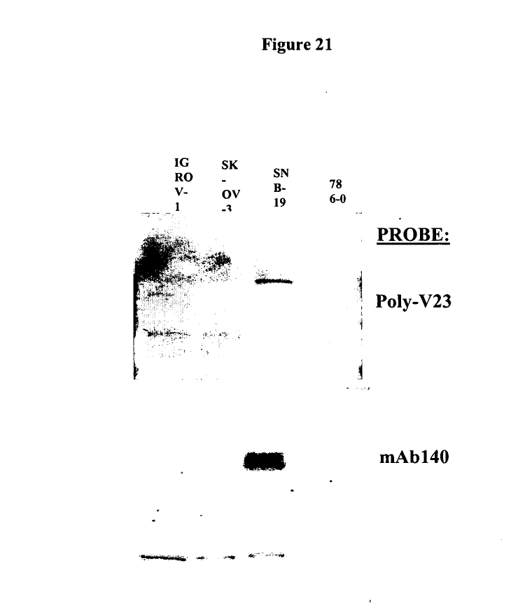

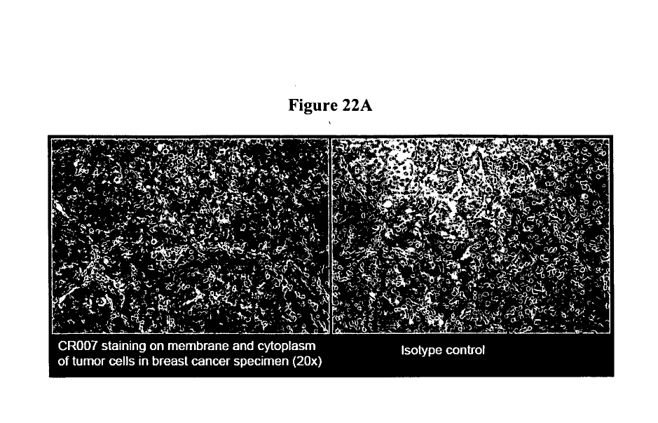

It was also determined that the anti Ten-M2 mAb could recognize endogenous Ten-M2 protein in cancer cells. Total cell lysates made from IGROV-1, SK-OV-3, SNB-19 and 786-0 cells were resolved by SDS-polyacrylamide gel electrophoresis and blotted to nitrocellulose membranes. Next, the western blots were incubated with either rabbit polyclonal antibody (FIG. 21, upper panel) generated against the Ten-M2 protein or anti-Ten-M2 antibody (FIG. 21, lower panel). A band around 300 kD that corresponds to the size of endogenous Ten-M2 protein was detected by both Ten-M2 rabbit polyclonal and monoclonal antibodies in SNB-19 cells. As a further control, no Ten-M2 protein was detected in transcript negative cell lines IGROV-1 and SK-OV3 cells.

Immunohistochemistry

Ten-M2 expression in various human cancer tissues was analyzed by immunohistochemistry using anti Ten-M2 mAbs. For immunohistochemistry, formalin fixed and paraffin embedded tissue sample sections derived from various human carcinoma tissues were stained with Ten-M2 mAbs. Antigen retrieval was performed with partial proteolysis by proteinase K (DakoCytomation, Carpinteria, Calif.) and endogenous peroxidase activity was quenched in a 3% solution of hydrogen peroxide in methanol.

Tissue sections were first blocked in a solution of 5% BSA (Sigma) and 1% goat serum (Jackson ImmunoResearch Lab) in PBS for 1 hr, then incubated with biotinylated TEN-M2 or biotinylated isotype control IgG₂ antibody diluted in blocking buffer. After 1 hr, the sections were washed and incubated with horseradish peroxidase conjugated streptavidin (1:200) for 45 min. The washing step was repeated, followed by development of stain using DAB reagent (Vector labs, Burlingame, CA). DAB reaction was stopped and the sections were counterstained in hematoxylin (Fisher Scientific), dehydrated and mounted with permount (Fisher Scientific).

As seen in Table 8 below, strong staining was observed on the membrane in breast, renal and prostate cancer (+2). Weaker positive staining with an intensity score of +1 or greater was also identified in most human breast, prostate, colon, endometrial, renal clear cell, lung, brain, ovarian carcinoma and lymphoma and melanoma specimens.

TABLE 8
Anti Ten-M2 Immunohistochemostry Summary

| | # of specimens w/highest staining | | | |
|---|---|---|---|---|
| | intensity seen | | | |
| Tissue | 3+ | 2+ | 1+ | 0 |
| Breast CA | 0 | 0 | 10 | 2 |
| Prostate CA | 0 | 3 | 7 | 0 |
| Colon CA | 0 | 0 | 7 | 3 |
| Endometrial CA | 0 | 0 | 9 | 1 |
| Renal CA | 0 | 4 | 5 | 0 |
| Lung CA | 0 | 3 | 7 | 0 |
| Brain CA | 0 | 0 | 4 | 6 |
| Ovarian CA | 0 | 0 | 9 | 1 |
| Lymphoma | 0 | 0 | 6 | 4 |
| Melanoma | 0 | 2 | 8 | 0 |
| Breast | 0 | 0 | 1 | 3 |
| Prostate | 0 | 0 | 5 | 0 |
| Colon | 0 | 0 | 1 | 3 |
| Uterus | 0 | 0 | 1 | 4 |
| Kidney | 0 | 0 | 4 | 0 |
| Lung | 0 | 0 | 1 | 5 |
| Ovary | 0 | 2 | 2 | 1 |
| Tonsil | 4 | 1 | 0 | 0 |
| Pancreas | 0 | 0 | 2 | 0 |
| Testis | 0 | 0 | 1 | 0 |
| Adrenal Gland | 0 | 0 | 1 | 0 |
| Parotid Gland | 0 | 0 | 1 | 0 |
| Stomach | 0 | 0 | 1 | 0 |
| Liver | 0 | 0 | 2 | 0 |
| Thyroid Gland | 0 | 1 | 2 | 0 |

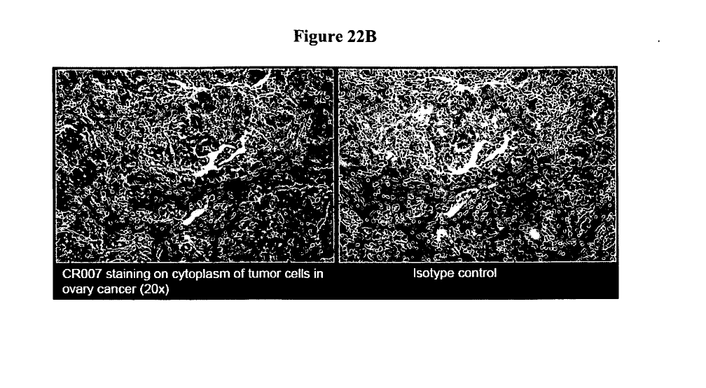

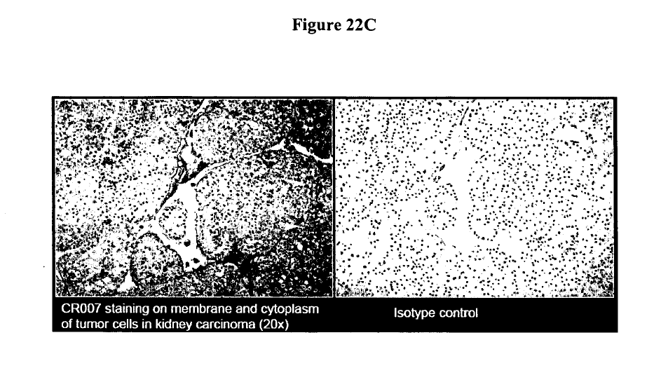

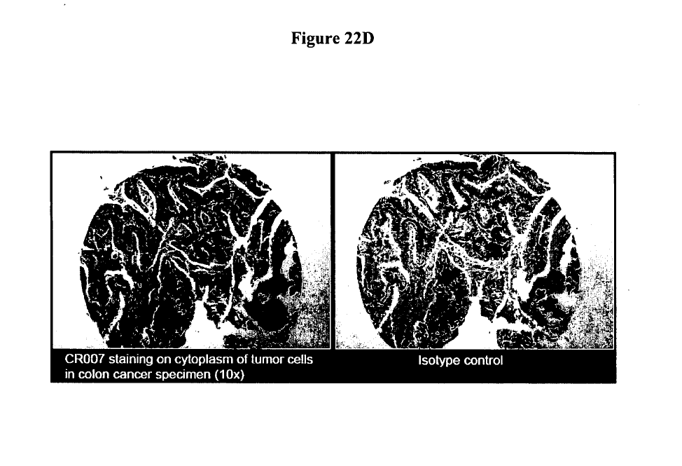

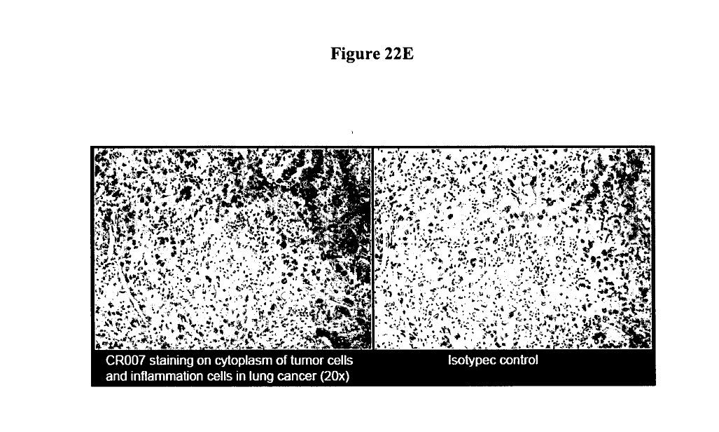

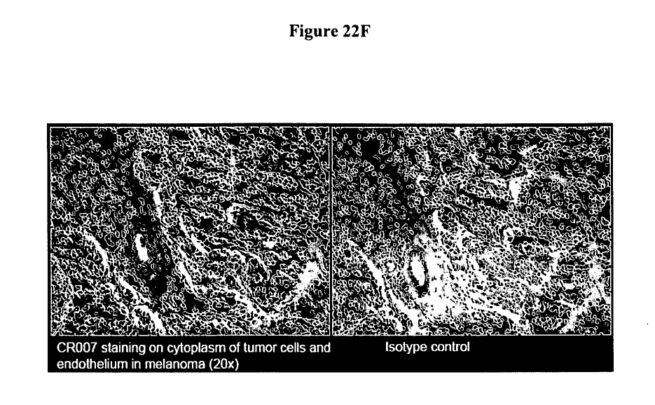

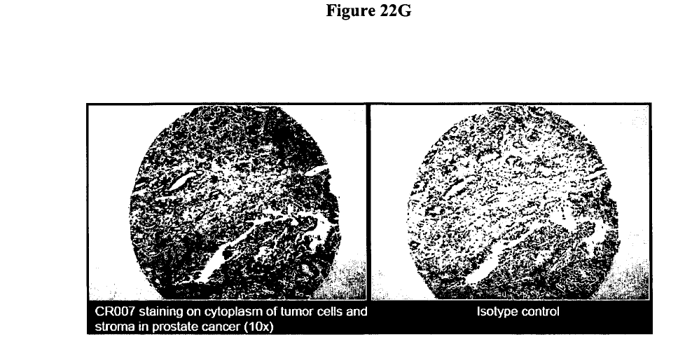

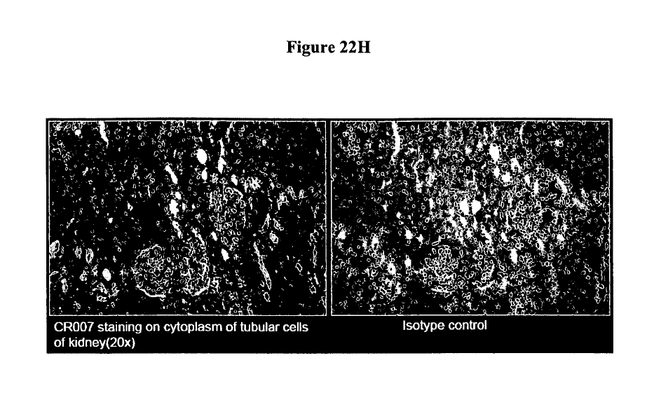

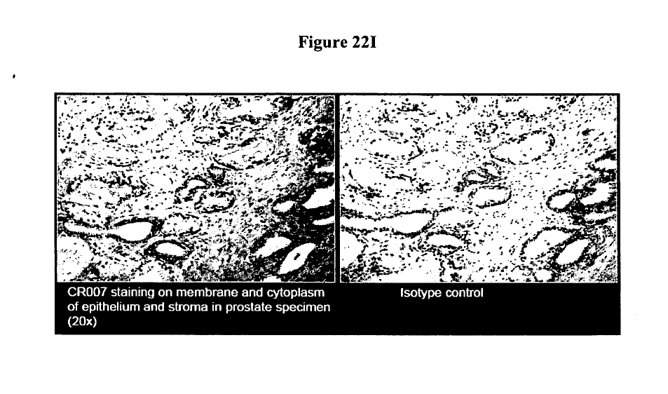

Examples of staining of breast, prostate, colon, renal clear cell, lung, ovarian carcinoma and melanoma are presented in FIG. 22 A-G respectively. Anti-Ten-M2 antibody staining revealed that the majority of the Ten-M2 protein is found on membrane and cytoplasm region of breast cancer, ovarian cancer, renal cell carcinoma, colon cancer, lung cancer, melanoma, and prostate cancer tumor cells. Interestingly, anti-Ten-M2 antibody also stained the endothelium of melanoma samples suggesting possible antiangiogenic opportunities, but did not stain the endothelium of a number of normal tissues. No cancer tissue staining was observed with control IgG. Immunohistochemical staining with anti-Ten-M2 antibody was also carried out on normal human tissues as listed in Table 8. Positive staining was found mainly in normal kidney, prostate, ovary, tonsil and thyroid gland. Of note, most tissue staining was cytoplasmic as shown on tubules of the kidney. Prostate gland showed membrane staining of the stroma (FIG. 22 H-I).

Flow Cytometry

Quantitative analysis of CG50426 (Ten-M2) expression on the surface of 15 different cell lines was determined by flow cytometry (FACS). Approximately 1 ×10⁶ cells were harvested, washed and incubated with a saturating amount (1 μg/ml) of either TEN-M2 or isotype-matched control antibody in staining buffer containing PBS (pH 7.4), 4% FBS and 0.1% NaN₃ for 30 min on ice, followed by washing and staining with R-Phycoerythrin (PE)-conjugated goat-anti-human antibody (Jackson ImmunoResearch Laboratories, Inc, West Grove, Pa.) at 1:100 for 30 min on ice. Cells were fixed in 1% paraformaldehyde/PBS and examined on a Becton Dickinson FACSCalibur flow cytometer. Data analysis was performed with Becton Dickinson Cell Quest software version 3.3 and the geometric mean fluorescence intensity ratio (GMR) was determined for each cell type.

As shown in Table 9 below, FACS analysis identified 7 cancer cell lines including SNB-19, RXF631, RXF393, 786-0, T47D, NCI-H82 and Hop62 cells that had surface staining with anti-Ten-M2 mAb with at least 3-fold above isotype control mAb background.

TABLE 9
Summary of RTQ PCR, FACS and in vitro growth inhibition
of human cancer cell lines with anti-Ten-M2-mAbs

| Brain | CT | GMR | ADC (IC50) |
|---|---|---|---|
| SNB-19 | 25 | 31 | +++ Anti-TEN-M2-vcMMAE: < 60 pM |
| | | | +++ Anti-TEN-M2-MMAF: < 60 pM |
| Kidney | CT | GMR | ADC |
| RXF-631 | 24 | 14 | ++ Anti-TEN-M2-vcMMAE: 7.6 nM |
| | | | +++ Anti-TEN-M2-MMAF: < 60 pM |
| RXF-393 | 26 | 4 | +++ Anti-TEN-M2-vcMMAE: 60 pM |
| | | | +++ Anti-TEN-M2-MMAF: 60 pM |
| 786-0 | 28 | 4 | + Anti-TEN-M2-vcMMAE: > 38 nM |
| | | | + Anti-TEN-M2-MMAF: > 38 nM |
| Caki-1 | 35 | 1 | — |
| A498 | 40 | 1 | — |
| Breast | CT | GMR | ADC (IC50) |
| BT549 | 40 | 1 | ND |
| T47D | 28 | 3 | ND |
| ZR75-1 | ND | 1 | ND |
| Lung | CT | GMR | ADC (IC50) |
| NCl-H82 | 26 | 4 | ND |
| Hop-62 | 28 | 4 | ND |
| NCl-H69 | 29 | 1 | ND |
| NCl-H522 | 33 | 1 | ND |
| Ovary | CT | GMR | ADC (IC50) |
| OVCAR-3 | 40 | 1 | — |
| IGROV-1 | 40 | 1 | — |

^{a} TEN-M2: CT values were determined by RTQ PCR as described in Materials and Methods. Geometric Mean ratios (GMR) were determined by flow cytometric analysis. Antibody-Drug Cytotoxicity (ADC) or cell killing was determined by clonogenic assay as described.
^{b} IC₅₀ value is the mean and SD of two independent clonogenic assays with each experiment performed in triplicate wells.
ND: Not done.

### Example 9

### In Vitro Growth-Inhibition of Brain Carcinoma and Renal Cell Carcinoma Cell Lines with Anti-Ten-M2-vcMMAE and Anti-Ten-M2-MMAF

To investigate whether anti-Ten-M2-vcMMAE and anti-Ten-M2-MMAF specifically inhibited the growth of antigen-positive cells, cell killing assays were performed to assess cell viability after anti-Ten-M2 drug conjugates treatment. Cells were plated in 96-well plates and allowed to recover overnight. Anti-Ten-M2-vcMMAE or Anti-Ten-M2-MMAF antibody conjugates at various concentrations was added to sub-confluent cell cultures, and incubated for 4 days at 37° C. The cells were then transferred into 6-well plates and allowed to grow for another 7 days. Since RXF631, RXF393 and 786-0 cells do not form colonies, cell counting method was used. Briefly, cells in each well were collected and resuspended into 50 μl of growth media. The number of cells was counted using hemocytometer under microscope. The surviving cell fractions were calculated based upon the ratio of the treated sample and the untreated control. The IC₅₀ was defined as the concentration resulting in a 50% reduction of colony formation or cell number compared to untreated control cultures.

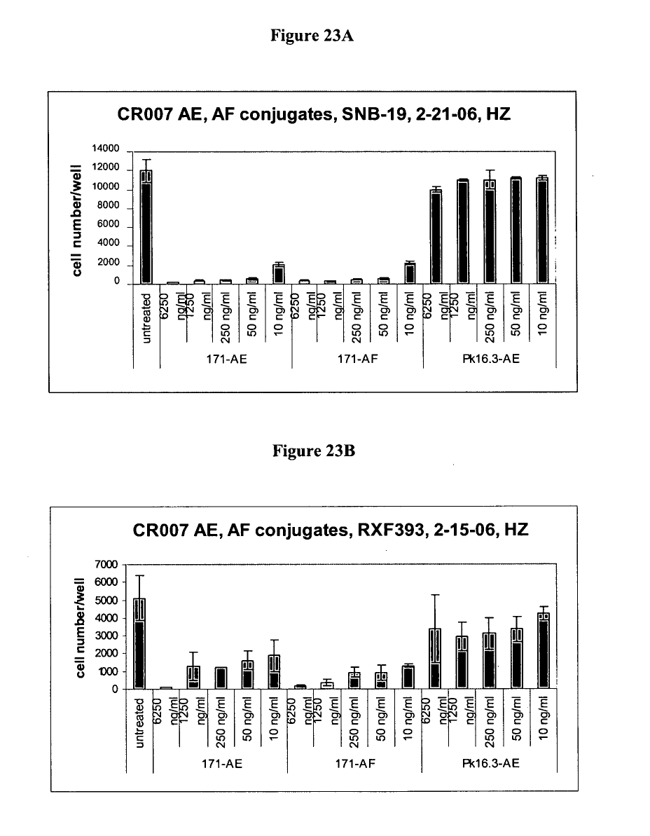

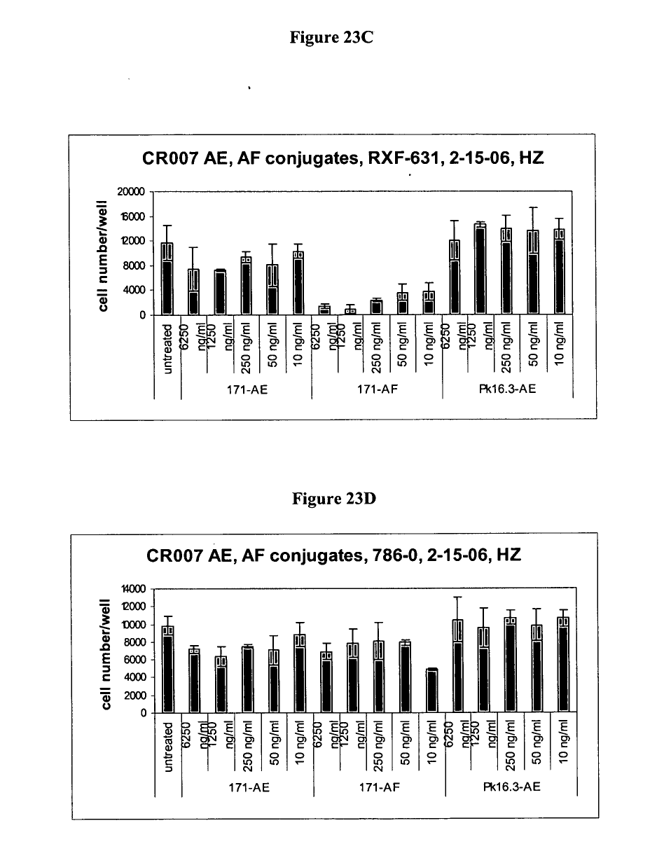

As shown in Table 9, Ten-M2 expressing cells were sensitive to growth-inhibition induced by anti-Ten-M2-vcMMAE and Anti-Ten-M2-MMAF, but not cells that did not express the antigen. The best killing effect of anti-Ten-M2 drug conjugates was observed on SNB-19 and RXF393 cells with IC₅₀ around 60 pM (FIG. 23A, B). RXF631 cells were more sensitive to Anti-Ten-M2-MMAF (IC₅₀<60 pM) than to anti-Ten-M2-vcMMAE (IC₅₀=7.6 nM) (FIG. 23C). Consistent with our previous observation that 786-0 was not sensitive to either free MMAE or free MMAF, anti-Ten-M2 mAb drug conjugates had little effect on 786-0 cell growth (FIG. 23D). Antibody PKl6.3 was conjugated to vcMMAE and used as a IgG control in the same experiment. It had about 30% non-specific growth inhibitory effect on RXF-393 cells, but showed no effect on all other 3 cell lines.

These data indicate that anti-Ten-M2 mAb conjugated to a drug such as MMAE or MMAF are highly potent and selective agents for the treatment of brain tumor and renal cell carcinoma.

### INCORPORATION BY REFERENCE

All references cited herein, including patents, patent applications, papers, text books, and the like, and the references cited therein, to the extent that they are not already, are hereby incorporated herein by reference in their entirety.

### EQUIVALENTS

The foregoing written specification is considered to be sufficient to enable one skilled in the art to practice the invention. The foregoing description and Examples detail certain preferred embodiments of the invention and describes the best mode contemplated by the inventors. It will be appreciated, however, that no matter how detailed the foregoing may appear in text, the invention may be practiced in many ways and the invention should be construed in accordance with the appended claims and any equivalents thereof.

What is claimed is:

1. A fully human monoclonal antibody, or a binding fragment thereof, that binds to Ten-M2 and neutralizes Ten-M2 activity.

2. The fully human monoclonal antibody of claim 1, wherein said antibody is a full-length antibody.

3. The fully human monoclonal antibody, or binding fragment thereof, of claim 1, wherein said antibody, or binding fragment thereof, binds to Ten-M2 with a K_{D} of less than 18 nM.

4. The fully human monoclonal antibody, or binding fragment thereof, of claim 1, wherein said antibody, or binding fragment thereof, binds to Ten-M2 with a K_{D} of less than 15 nM.

5. A human monoclonal antibody that binds to Ten-M2 and comprises a heavy chain having an amino acid sequence selected from the group consisting of SEQ ID NOS: 2, 6, 10, 14, 18, 22, 26, 30, 34, 38, 42, 46, and 50.

6. The antibody of claim 5, further comprising a light chain having an amino acid sequence selected from the group consisting of SEQ ID NOS: 4, 8, 12, 16, 20, 24, 28, 32, 36, 40, 44, 48, and 52.

7. An antibody immobilized on an insoluble matrix, wherein the antibody is the antibody of claim 1.

8. A method for assaying the level of Ten-M2 in a patient sample, wherein said method comprises the steps of:
(a) contacting the patient sample with the anti-Ten-M2 antibody of claim 1; and
(b) determining the presence or amount of anti-Ten-M2 antibody bound to Ten-M2,
thereby detecting the level of Ten-M2 in said patient sample.

9. The method according to claim 8 wherein the patient sample is blood.

10. A composition comprising the antibody of claim 1, or a binding fragment thereof, and a pharmaceutically acceptable carrier.

11. A method of treating malignant tumors, comprising administering to an animal in need thereof a therapeutically effective dose of an antibody that specifically binds to Ten-M2, or a binding fragment thereof.

12. The method of claim 11, wherein said animal is human.

13. The method of claim 11, where said antibody is a fully human monoclonal antibody.

14. The method of claim 11, wherein said malignant tumor is selected from the group consisting of: lung, kidney, brain, and ovary.

15. The method of claim 11, wherein the antibody is the antibody of claim 1.

16. An antibody, or binding fragment thereof, that binds to Ten-M2, wherein said antibody, or binding fragment thereof, neutralizes a Ten-M2-induced activity, and wherein said antibody, or binding fragment thereof, cross-reacts with a fully human anti-Ten-M2 antibody selected from the group consisting of Mab120, Mab140, and Mab171, Mab179, Mab 199, Mab 213 or an antibody in the same antigen-binding bin as fully human anti-Ten-M2 antibody Mab120, Mab140, and Mab171, Mab179, Mab199, or Mab 213.
